(19) United States
(12) Patent Application Publication
(10) Pub. No.: US 2014/0193440 A1
(43) Pub. Date: Jul. 10, 2014

(54) MARKERS OF ALZHEIMERS DISEASE

(71) Applicant: ImmunoClin Ltd., London (GB)

(72) Inventors: Mario Clerici, Milan (IT); Giorgio Annoni, Milan (IT); Beatrice Arosio, Lissone (IT)

(21) Appl. No.: 14/149,362

(22) Filed: Jan. 7, 2014

Related U.S. Application Data
(63) Continuation-in-part of application No. 13/356,374, filed on Jan. 23, 2012, now abandoned.
Application No. 13/356,374 is a continuation of application No. 10/516,421, filed on Jun. 30, 2005, now abandoned.

(30) Foreign Application Priority Data
Dec. 11, 2013 (GB) 0212648.0

(51) Int. Cl. C12Q 1/68

(52) U.S. Cl. CPC C12Q 1/6883
USPC 424/184.1

(57) ABSTRACT

An inflammatory process is suggested to be involved in the pathogenesis of Alzheimer's disease (AD), a neurodegenerative disorder characterized by the presence of neuritic plaques within the cerebral cortex that are mainly composed of a small insoluble protein of 40-42 aminoacids (amyloid protein). Amyloid-specific Interleukin-10 (IL-10) generation is found to be selectively and significantly reduced in AD patients (p=0.023). The genotype associated with high IL-10 production is extremely infrequent in AD individuals (2% vs. 28%). The presence of low/intermediate-IL-10-producing genotypes (GCC/ATA; ATA/ATA) was associated with an earlier age at disease onset and (ACC/ACC; ACC/ATA) with an accelerated rate of disease progression/severity and with amyloid-specific impairment of IL-10 production. This relationship is independent of ApoE gene polymorphism. These results support the use of anti-inflammatory compounds in the therapy of this disease.

### RELATED APPLICATIONS

This application is a continuation-in-part of U.S. patent application Ser. No. 13/356,374 filed on Jan. 23, 2013 and titled TREATMENT WITH CYTOKINES, which is a continuation of U.S. patent application Ser. No. 10/516,421, filed Jun. 30, 2005, and titled TREATMENT WITH CYTOKINES, which is a U.S. national phase application, pursuant to 35 U.S.C. §371, of PCT international application Ser. No. PCT/GB03/02369 filed May 30, 2003, designating the United States, and published in English as international publication WO 03110223 7 Al on Dec. 11, 2003, which claims priority to GB 0212648.0 filed May 31, 2002. The entire contents of the aforementioned patent applications are incorporated herein by this reference.

### FIELD OF THE INVENTION

At present definitive diagnosis of Alzheimer's disease (AD) is only possible at post-mortem hence research efforts have mostly focused on finding biomarkers that could help diagnosing AD. Search for prognostic markers to identify people at risk of developing AD is a relatively recent trend triggered by novel scientific advances suggesting that connecting biomarkers to AD risk is possible. Alzheimer disease (AD) as currently classified has several forms, of which two are relevant to genetic testing: A very small percentage of AD cases (5-7%) arise in family clusters with early onset (before the age of 60) and the remaining majority of cases (>90%) with late onset (after the age of 60).

Current state of knowledge suggests that familial early-onset AD (EOAD) is caused by a dominant mutation in one of three genes: PSEN1, PSEN2, or APP. A person with one of these mutations is at risk of developing symptoms before age 60. Such families are quite rare, but the 50 percent risk for each child of an affected member to carry the causative mutation means that these tests can be important for those at risk. In contrast to early onset AD, variants of the APOE gene confer increased risk of developing the late onset form of AD (LOAD) most commonly seen in the general population (accounts for vast majority of AD cases). Patents relevant to genetic testing for all four genes have been granted in the United States to Duke University and then licensed to Athena Diagnostics. Multiple companies offer predictive testing for nuclear polymorphism that have been shown to be associated with AD and these include genetic testing for PSEN1, PSEN2, APP, and APOE.

CPKO has licensed technology platform to Bristol Myers Squibb in which peptoid libraries are screened against serum samples from patients. Three peptoids were identified that captured at least 3-fold higher levels of IgG antibodies in AD patients using this platform suggesting that assessment of such antibody level could be used as diagnostic test. This has been demonstrated in a very small group of patients with control patients also showing high levels of the antibodies (2 out of 8 tested). It is not clear whether this signifies non-specific false positive or whether such patients could be at risk of developing AD. Additional studies required for regulatory approval and commercial use of such tests are being carried out in addition to and phylogenetic analysis to uncover such genetic associations in other diseases, including cancer, autism, diabetes, infectious diseases and chronic obstructive pulmonary disease (COPD). DiaGenics developed test to detect AD based upon expression of 96 genes; the test has been licensed by Pfizer and has been CE marked and is being made available to the market while development is ongoing. It is suggested that patients with both positive and negative ADtect tests are screened for clinical signs of the disease using standard clinical practice so it is not yet clear to what extend this test aids clinical diagnosis and management.

Zinfandel Pharmaceuticals and Takeda signed license agreement in 2011 to develop TOMM40 as a companion diagnostic for ACTOS, TAKEDA's anti-diabetic drug with a potential to minimized AD (based upon small set of data). After initial trial in AD patients who were diabetic, ACTOS activity was not confirmed in AD patients who do not suffer from diabetes. ACTOS has been taken off the market in May 2011 in some countries due to carcinogenic activity and further research is being conducted to understand and improve activity of ACTOS like drugs before companion diagnostics are developed.

The outcome of predictive testing is to develop AD predictive test personalized test using miniaturized device to provide rapid answer within 1-2 h of testing, flexible towards modifications of genetic polymorphism patterns to enhance the performance of the prognostic tool.

At present the most predictive and clinically used in late onset AD (LOAD) is testing for ApoE polymorphism but while patients with certain ApoE polymorphisms are at risk, the presence of such polymorphism per se does not guarantee the development of the disease (similarly to the routinely used cardiovascular markers such as cholesterol or CRP: high level signifies the risk but does not guarantee the development of cardiovascular disease or accurately predict cardiovascular event like a heart attack). what occur Our test assesses genetically coded inflammatory components that contribute to late onset AD (LOAD) development in addition to assessing risk due to ApoE polymorphism.

This invention relates to the use of markers that participate in inflammatory processes and are associated cytokines in the diagnosis, treatment or prophylaxis of diseases. More particularly, the present invention relates to the use of cytokines to diagnose or treat non-neoplastic or non-leukaemic diseases such as autoimmune diseases or neurodegenerative disorders.

In the description which follows, the present invention will be described with particular reference to the most preferred embodiment of the invention which relates to the use of the cytokine interleukin-10 in the diagnosis, treatment or prophylaxis of the neurodegenerative disorder Alzheimer's disease. It is not intended to restrict the scope of the present invention to this one embodiment since the present invention finds equal utility in other disorders such as autoimmune diseases, for example multiple sclerosis, myasthenia gravis, systemic lupus erythematosus, diabetes mellitus and asthma, other neurodegenerative disorders for example Parkinson's disease, motor neurone disease and Alzheimer's disease; chronic inflammatory diseases such as rheumatoid arthritis; and other diseases where the modulation of T-Cell function is desirable such as HIV-infection and AIDS.

Similarly, the invention has utility with all cytokines, not solely interleukin-10 and hence it is intended to include cytokines such as interleukin-1 (α or β), interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β, in the scope of the present invention.

The major cause of cognitive decline in the elderly is Alzheimer's disease (AD). Because of longer life spans worldwide, the number of people that will be affected by AD is expected to triple over the next 50 years (1). AD is a clinical syndrome characterised by complex and heterogeneous pathogenic mechanisms. The recognised genetic factors include mutations of the gene encoding the amyloid precursor protein (2), presenilin 1 and 2 (3, 4), which account for a small part of familial and usually early-onset AD cases. Genetic factors have also been associated with the sporadic or non-familial form of the disease and the allele e4 of the apolipoprotein E (Apo E) significantly increases the risk of AD, but is neither necessary nor sufficient for the development of the disease (5-7). Therefore other genetic and environmental factors are likely to be implicated and are actively investigated.

Molecules that take part in the inflammatory cascade are of great interest, because inflammation has repeatedly been suggested to be associated with the neurodegenerative process characteristic of the AD brain (8). Thus, reactive astrocytosis is observed both in the cortex and in the hippocampus of these patients and the glial cells are also activated within or near the neuritic plaques. Over-expression of cytokines and other inflammatory molecules are common features of the AD brain pathology (9). Additionally, epidemiological studies showed that the long term use of non-steroid anti-inflammatory drugs is associated with a decreased incidence of AD in a co-twin control study (10) and several other clinical studies confirmed a decreased association of AD in individuals treated with anti-inflammatory drugs (11) including COX2 specific inhibitors (12). These findings support the hypothesis that inflammation might contribute to the neurodegeneration associated with AD (13).

In the attempt to better understand the biology of AD the possible role of several cytokines and chemokines has recently been investigated. Virtually all of the mediators analyzed in these studies, including IL-1b, IL-6, TNF-α, IL-8, TGF-β and macrophage inflammatory protein-1a (MIP-1a), have been suggested to be up-regulated in AD compared to non demented controls (14-18). On the contrary, conflicting results are obtained in relation to the immunomodulatory cytokine IL-10, a type-2 cytokine that suppresses T lymphocytes and cell-mediated immunity in humans and mice and has potent anti-inflammatory properties (19-21).

These studies considered each cytokine independently as gene polymorphisms and/or production, but never investigated the relationship between factors acting for and against inflammation, such as IL-10 and IL-6, in the same population sample.

It is worth recalling that single nucleotide polymorphisms (SNPs) in the promoter region of these two genes are known. The gene encoding IL-10, mapped to chromosome 1 between 1q31 and 1q32, is highly polymorphic. IL-10 production is correlated to biallelic polymorphisms at positions: −1082 (guanine to adenine substitution), −819 (thymine to cytosine substitution), and −592 (adenine to cytosine substitution). The polymorphism at position −1082 lies within an Ets (E-twenty-six specific)-like recognition site and may affect the binding of this transcriptional factor and therefore alter transcription activation; the −1082 A allele correlates with IL-10 generation after stimulation of T cells in vitro (57), while polymorphisms at positions −819 and −592 do not seem to be involved. The IL-6 gene in humans is organised in five exons and four introns and maps to the short arm of chromosome 7 (7p21) (50, 73). The involvement of IL-6 in many biological functions is paralleled by genetic associations of its allelic variants with several physiological and pathophysiological conditions. Two of its polymorphic sites have been frequently used for genetic association studies: a multiallelic variable number of tandem repeats (VNTR) polymorphism in the 3′ flanking region (AT repeats) and a biallelic G-to-C polymorphism of the promoter at the position −174. The G/C single nucleotide polymorphism (SNP) seems to be associated with varying blood levels and transcription rates of IL-6 (54, 56, 68).

In the light of these considerations and on the basis of a case-control association study in Italian sporadic late-onset AD patients and matched healthy controls (HC), the present inventors evaluated whether IL-10 and IL-6 SNPs were related with the development of AD. The results shed further light on the inflammatory pathogenic hypothesis of AD and suggest an independent genetic predisposition from the metabolic one.

These allelic variations are associated with measurable differences in IL-10 and IL-6 production by antigen- and mitogen-stimulated peripheral blood lymphocytes. In fact, these polymorphisms occur in the regulatory region of the gene and are associated with high, intermediate or low IL-10 production (22).

The present inventors investigated beta amyloid-stimulated IL-10 and IL-6 production by peripheral blood lymphocytes (PBMC) of AD patients and of age-matched healthy controls. Because the generation of this cytokine was significantly reduced in AD patients, IL-10 polymorphisms were analysed in these same individuals. Results showed that the high IL-10-producing allele is extremely rare in AD patients.

Specifically, IL-10 genotypes are differently distributed when AD are compared with HC (χ2=16.007; p=0.007). Therefore genotypes corresponding to reduced IL-10 production have a significantly higher distribution amongst AD subjects (table I). The presence of low-IL-10-producing genotypes is associated with a worsened clinical outcome of AD as follows: 1) earlier age at disease onset (Table II); and 2) faster disease progression (MMSE score)(Table III).

TABLE I
IL-10 genotype distribution

| | AD | HC | | |
|---|---|---|---|---|
| Genotype (c) | n = 47 | n = 25 | AD % | HC % |
| GCC/GCC (H) | 1 | 7 | 2 | 28 |
| GCC/ACC (M) | 10 | 9 | 21 | 36 |
| GCC/ATA (M) | 11 | 3 | 23 | 12 |
| ACC/ACC (L) | 8 | 1 | 17 | 4 |
| ACC/ATA (L) | 12 | 4 | 26 | 16 |
| ATA/ATA (L) | 5 | 1 | 11 | 4 |

The frequency of the different genotypes among Alzheimer's disease patients (AD) are statistically different from those of the healthy controls (HC). χ²=16.007, df=5, p=0.007. In the brackets (c) there are the corresponding phenotype high (H), intermediate (M), low (L).

TABLE II
IL-10 genotype distribution and age at onset

| Genotype | mean | S.D. | SEM |
|---|---|---|---|
| GCC/GCC | 76 | / | / |
| GCC/ACC | 75.00 | 8.57 | 3.03 |
| GCC/ATA | 67.33 | 8.2 | 2.73 |
| ACC/ACC | 76.20 | 8.79 | 3.93 |
| ACC/ATA | 77.17 | 4.07 | 1.66 |
| ATA/ATA | 65.75 | 1.71 | 0.85 |

Correlation between the different genotypes in Alzheimer's disease patients and the age at onset. ANOVA: p=0.042.

TABLE III
IL-10 genotype distribution and MMSE

| Genotype | mean | S.D. | SEM |
|---|---|---|---|
| GCC/GCC | 18 | | |
| GCC/ACC | 21.75 | 5.5 | 1.94 |
| GCC/ATA | 16.33 | 5.68 | 1.89 |
| ACC/ACC | 10.80 | 7.5 | 3.35 |
| ACC/ATA | 13.83 | 5.19 | 2.12 |
| ATA/ATA | 22.5 | 1.73 | 0.87 |

Correlation between the different genotypes in Alzheimer's disease patients and MMSE ANOVA: p=0.010.

Chronic inflammation is suggested to be involved in the neurodegenerative process characteristic of AD (24, 25); this suggestion stems from both in vivo and ex adjuvantibus criteria. Hence, inflammatory mediators and activated glial cells are observed to be closely associated with neuritic plaques in vivo. Furthermore, recent data indicate that anti-inflammatory therapy could be useful in modulating disease progression (10-12). Despite this vast body of evidence, the biologic correlates of AD are still unclear. To shed light on this problem, attention was focused on IL-10. This cytokine is a pivotal regulatory cytokine involved in many facets of the immune response and is dysregulated in human autoimmune (26), malignant (27-31), and infectious (32-35) diseases. More recently it has been shown that the presence of genetically-determined higher levels of IL-10 secretion is an important component of the genetic background to systemic lupus erythematosus (36) and to the outcome of infectious disease (37). It has also been demonstrated that IL-10 secretion, resulted from in vitro stimulation of human peripheral blood leukocytes with LPS, varies markedly between individuals and that cytokine haplotypes are associated with different secretion levels (38). In addition, differences in IL-10 serum production by cells of patients and of their first-degree family members (37, 39), as well as differences in the distribution of IL-10 alleles, suggested the involvement of the different isoforms of the IL-10 gene as an important quantitative trait loci for human disease in infections (37, 40), autoimmune (26, 36, 41, 42) and malignant diseases (43).

The present inventors initially analyzed LPS-, Flu-, and amyloid peptide-specific IL-2 and IL-10 production by peripheral blood mononuclear cells (PBMC) of AD patients and age matched HC. Results showed that: 1) IL-2 production by PBMC of AD patients and controls was similar in all the conditions measured; and 2) IL-10 generation by LPS- and Flu-stimulated PBMC was comparably similar amongst the two groups of individuals. In contrast, an amyloid-specific immune impairment characterized by a reduced generation of IL-10 was present in AD. The observation that this cytokine imbalance was not seen in mitogen-stimulated PBMC indicates that amyloid-specific immune responses are selectively impaired in AD patients. Additionally, results showing that flu-stimulated proliferation was similar in patients and controls indicates that antigenic processing and presentation in association with HLA class II molecules, and the CD4-HLA class II self-restricted pathway of activation of the immune system (44), are not defective in these patients.

Next polymorphisms were analyzed in the IL-10 gene in the same group of subjects. Results stemming from analysis of the distribution of the IL-10 alleles in this Italian sample of healthy individuals showed a close similarity to those reported for other caucasian populations (45). In contrast, we observed a significantly higher frequency of the genotypes corresponding to reduced IL-10 production (ACC/ACC, ACC/ATA and ATA/ATA) in AD patients. Thus, an abnormally augmented prevalence of low-IL-10 producing isoforms in the AD population was determined; the phenotypic correlate of these isoforms becomes evident when amyloid-specific immune responses were measured.

Subsequent analyses focused on possible correlations between impaired IL-10 production and the clinical manifestations of AD by verifying whether the presence of low/intermediate IL-10 producing genotypes would be are associated with different disease outcomes. Results confirmed this to be the case. Thus, the presence of the ATA/ATA and of the GCC/ATA genotypes was correlated with an earlier age at disease onset. Additionally, the ACC/ATA and the ACC/ACC (all these are low/intermediate IL-10-producing genotypes) alleles were associated with a more severe cognitive impairment as indicated by a lower MMSE score.

It is interesting to observe that a recent report on Italian centenarians, individuals who—by definition—are less prone to develop age-related diseases, has demonstrated that extreme longevity is associated with a significantly higher frequency of the high IL-10-producing genotypes (46).

IL-10 is known to have potent anti-inflammatory properties (47); a biological scenario could thus be hypothesized in which the reduction of amyloid-specific IL-10 production would favour the triggering of the chronic inflammatory process seen in the progression of AD. These results suggest that an amyloid-specific and IL-10-mediated inhibitory feed-back circuit may be active in non-AD individuals; the rupture of this circuit could be associated with or predictive for the development of AD. Recently, a convincing study showed that an IL-10/pro-inflammatory circuit that revolves around cells of the innate immune system regulates susceptibility to autoimmune diseases (48). These results are expanded by showing that an alteration of this circuit is present in AD patients.

The present inventors have identified polymorphic regions, which polymorphs are indicative of a dysfunction of cytokine production and hence are associated with a predisposition towards an autoimmune, neurodegenerative or chronic inflammatory disease.

At present, Alzheimer's disease is diagnosed by recognised criteria such as DMS IV or NINCDS-ADRDA (23), often in conjunction with a magnetic resonance image (MRI) or computer aided tomography (CT) scan of the brain to identify the characteristic amyloid plaques and neurofibrillary tangles together with atrophy of the hippocampal area of the brain.

A definitive confirmatory diagnosis of Alzheimer's disease is only possible by a visual inspection of the affected areas of the brain during a post-mortem examination or via brain biopsy (not recommended due to lack of effective therapies).

Therapies and methods for monitoring of Alzheimer's disease are being urgently sought. As the progress is made in efforts to prevent or delay neurodegeneration and disease progression, early detection of Alzheimer's and identification of susceptible patients will gain importance as this will allow preventive measures being employed as early as possible. Therefore a need exists to be able to provide predictive and reliable tests for susceptibility to Alzheimer's disease without the need for lengthy and subjective assessments of cognitive performance.

### SUMMARY OF THE INVENTION

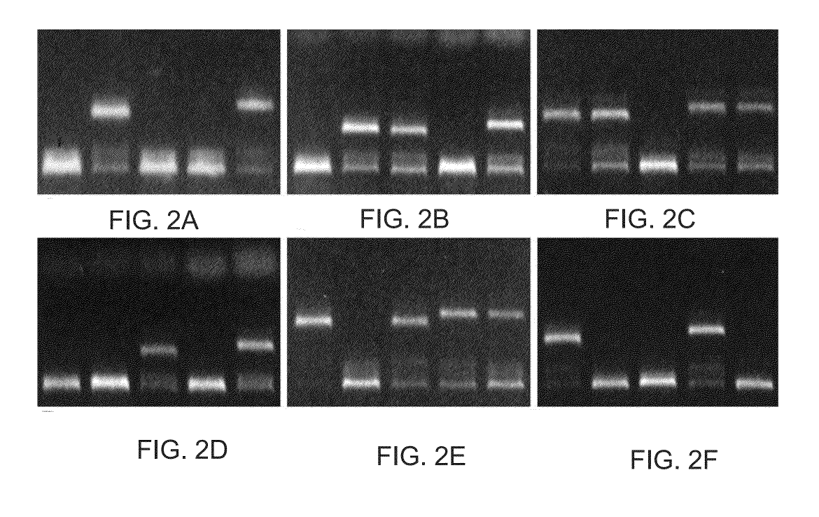

Accordingly, the present invention provides a method of determining the existence of or a predisposition to Alzheimer's disease, autoimmune disease or other neurodegenerative diseases, the method comprising the steps of taking a DNA bearing sample from a subject animal and analysing the sample to determine the allelic variants present at one or more of the SNP loci at positions −1082, −819 and −592 of the gene encoding IL-10, or to put it another way, analysing the sample for the presence or absence of the alleles of FIG. 2.

Preferably, the genotype at all three positions −1082, −819 and −592 is determined.

While the identification of the alleles of FIG. 2 has been found to be useful or predictive in the identification of Alzheimer's disease, a combination of the alleles of IL-10 and IL-6 has been found to be more strongly predictive of a predisposition to Alzheimer's or diagnostic of the presence of Alzheimer's disease.

Apolipoprotein E (Apo-E) has been associated with sporadic or non-familial AD. Hence, in a further aspect of the invention, a method of diagnosing Alzheimer's disease comprises the steps of obtaining a DNA-bearing sample from an animal and identifying the presence of a polymorphic allele of IL-10, IL-6 and of Apo-E.

Preferably, the polymorphic allele is one of the alleles of FIG. 2.

Additionally, the sample may be assayed for the presence/absence of polymorphisms or other allelic variations of other cytokines in addition to IL-10 and IL-6, for example, IL-10 and IL-6 plus IL-4 and/or IL-1.

Alternatively, the sample may be assayed for the presence of absence of polymorphisms or other allelic variations of IL-10 plus Apo-E, or IL-6 plus Apo-E.

An interleukin 1 alpha (IL-1 alpha) polymorphism has been associated with Alzheimer's disease (77). Hence, in still further aspect of the invention, a method of diagnosing Alzheimer's disease comprises the steps of obtaining a DNA-bearing sample from an animal and identifying the presence of a polymorphic allele of IL-10, IL-6, Apo-E and of IL-1.

Generally, optimal predictive value will be obtained by combining as many predictive factors as possible in the test. The methods described herein together with markers such as Apo-E and IL-1 enable the development of a powerful diagnostic method that would include all the biological markers shown to have a predictive value toward the development of AD.

The invention also provides a method of treating Alzheimer's disease, autoimmune diseases or other neurodegenerative disorders by modulating, that is augmenting or decreasing, the function of a gene having one of the allelic polymorphisms of IL-10 shown in Table I, or to put it another way, a gene of the allelic polymorphisms of FIG. 2.

For example, IL-6 production is preferably downregulated but IL-10 production is preferably upregulated. More preferably, IL-6 production is downregulated simultaneously with IL-10 production being upregulated.

Alternatively, pharmaceutical compositions which inhibit or supply the appropriate cytokines may be administered to a patient in need of treatment. For example, instead of down regulation of IL-6 at a genetic level, a patient may be supplied with compounds which inhibit or block the action of IL-6. This inhibition or blocking may be at the synthesis stage, at the site of action or anywhere along the IL-6 metabolic pathway. Similarly, IL-10 may be supplied directly, as an intermediate, as a pre-cursor or pre-pro-cursor, by stimulating the synthesis of IL-10 ab initio or by administration of pharmacological compositions that enhance or inhibit antigen specific production of interleukin-10 and, optionally, one or more other cytokines.

The other cytokine is preferably selected from the group consisting of interleukin-1 (α or β), interleukin-2, interleukin-3, interleukin-4, interleukin-5, interleukin-6, interleukin-7, interleukin-8, interleukin-9, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β.

Pharmacological agents which can modulate cytokine production are known in the art, for example, heat shock protein (HSP) and/or CpG-motif containing immunomodulatory oligonucleotides. DNA vaccination with constructs encoding the 60-kDa heat shock protein human hsp60 (phsp60) results in increased IL-10 production (71). It has been shown that CpG-DNA can induce the synthesis of suppressor of cytokine signalling (SOC) proteins. GpG-DNA-induced SOC proteins inhibit IL-6 production (72). Additionally, CpG-DNA via the extracellular signal-regulated kinase (ERK) mediated pathway, has been shown to trigger IL-10 production (73). CpG oligonucleotides can be structurally modified to achieve a desired profile of cell types affected and cytokines stimulated; to lean either toward the Th1 (cell mediated, interferon gamma generating) or Th2 (antibody, IL-10 and 11-4 generating) T helper cell pathway (74). Examples of such diverse modulations are: Th1 profiled compound 7909 generated by Coley Pharmaceuticals and Th2 profiled compounds generated by Dynavax (75). In addition, CpG-like immunomodulatory oligonucleotides in which CpG motif has been substituted with YpG or CpR motifs but which show promise of modification of their immunomodulatory potential via their chemical structure may also be employed as pharmacological agents to affect desired cytokine production profile (76).

In a further aspect, the present invention provides a method of treating Alzheimer's disease in an animal in need of treatment, the method comprising the reduction of IL-6 synthesis simultaneously with the augmentation of IL-10 synthesis.

The invention also provides the use of IL-6 inhibitors and IL-10 promoters in the manufacture of a medicament for the treatment of prophylaxis of Alzheimer's disease.

In a further aspect of the invention DNA fragments and cDNA fragments encoding the allelic polymorphism of Table I, or to put it another way the allelic polymorphisms of FIG. 2, for use in the above described method.

These DNA fragments are useful in the screening and identification of compounds which bind to, regulate, or otherwise have a modulatory effect these alleles and hence stimulate or inhibit the synthesis of the gene product.

Accordingly, the present invention further provides a method of screening for compounds which modulate chemokines implicated in Alzheimer's disease, the method comprising introducing the compound to be screened to DNA or cDNA fragments encoding the allelic polymorphisms of Table I, or to put it another way the allelic polymorphisms of FIG. 2 and assessing the hybridisation between the compound and the fragment.

Hence, the present invention also provides compounds which modulate Alzheimer's disease, as identified by the above method.

Preferably, the animal is a mammal and more preferably a human being.

The data presented herein support the role of inflammatory processes in the pathogenesis of AD; reinforce the hypothesis that in AD patients neurodegeneration is tightly associated with an aberrant antigen-specific immune response; and lend further support to the use of anti-inflammatory compounds in the therapy of this disease.

Accordingly, in a still further aspect the present invention provides a pharmaceutical composition comprising a cytokine in the preparation of a medicament for the treatment or prophylaxis of disease excluding neoplastic diseases, leukaemias, and acute inflammation. Preferably the disease is a neurodegenerative disorder or an autoimmune disease. Most preferably the disease is selected from the group comprising multiple sclerosis, myasthenia gravis, systemic lupus erythramatosus, diabetes mellitus, asthma, Parkinson's disease, motor neurone disease, Alzheimer's disease, chronic inflammation rheumatoid arthritis, HIV-infection and AIDS.

Preferably, the cytokine is selected from the group consisting of interleukin-1 (α or β), interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β, or combinations or mixtures thereof. Preferably, two or more cytokines are used.

Most preferably the ______ or each cytokine is an interleukin, especially interleukin-10 or interleukin-6.

Embodiments of the invention will now be described by way of example only, with reference to the accompanying drawings of which

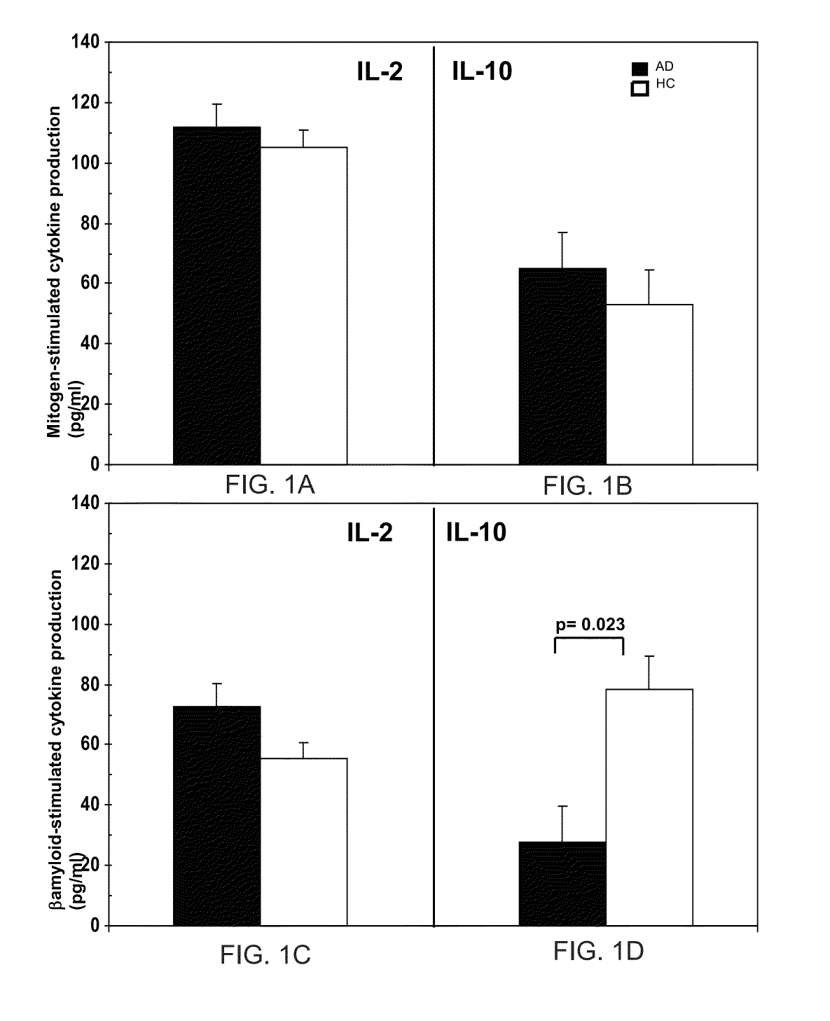

FIGS. 1A-1D are bar charts in which show LPS- and βamyloid- (a pool of 3β amyloid peptides: βA: fragments 25-35; βA: fragment 1-40; and βC: fragment 1-16) stimulated IL-2 (panels A and C) and IL-10 (panels B and D) production by PBMC of 47 AD patients (O) and 25 age- and sex-matched healthy controls (O). Mean values+standard errors are shown. p≦0.023.

FIG. 2 shows paradigmatic example of IL-10 genotyping for six different samples. In each gel the heaviest bands correspond to the amplicons of the human β-globin gene which is used as the internal controls. The other specific amplified DNA fragments correspond to the polymorphisms of the IL-10 gene: GCC/GCC (A), GCC/ACC (B), GCC/ATA (C), ACC/ACC (D), ACC/ATA (E), ATA/ATA (F).

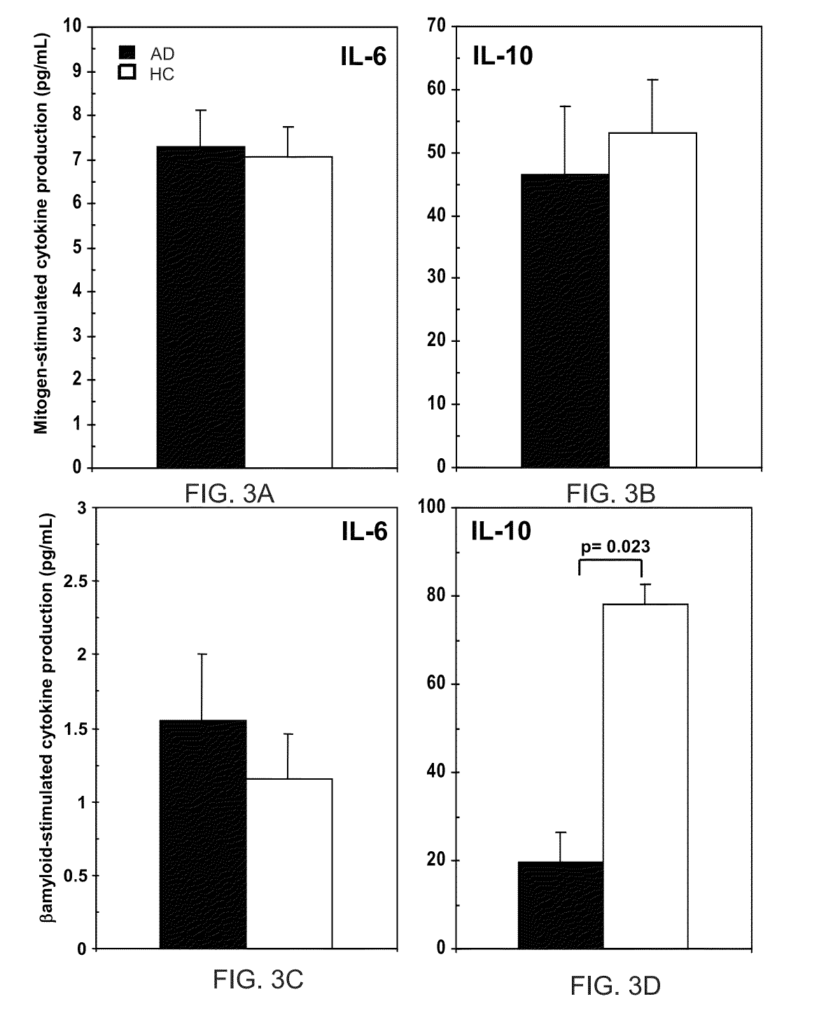

FIGS. 3A-3D are bar charts which show LPS- and β-amyloid-stimulated (a pool of three β-amyloid peptides; βA, fragment 25-35; βB, fragment 1-40; and βC, fragment 1-16) production of IL-6 (panels A and C) and IL-10 (panels B and D) by PBMC of 47 AD patients (O) and 25 age- and sex-matched healthy controls (O).

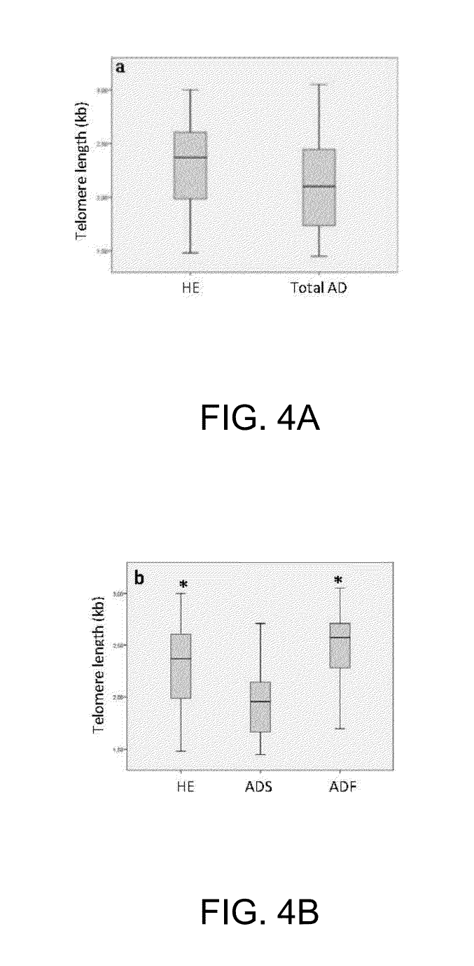

FIG. 4A depicts telomere length for HE and total AD.

FIG. 4B depicts telomere length for HE, ADS and ADF.

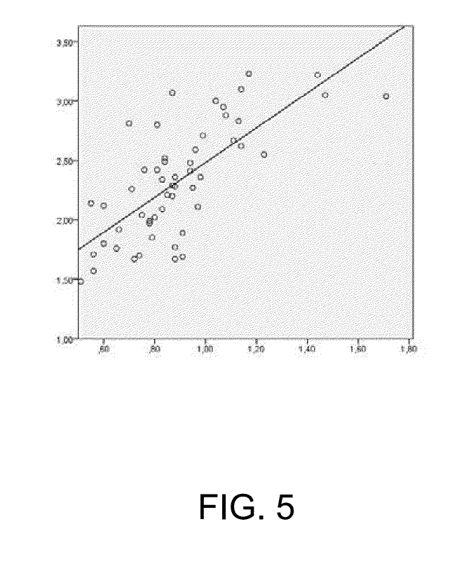

FIG. 5 depicts low cytometry TLs (kb) to q-PCR TLs (T/S).

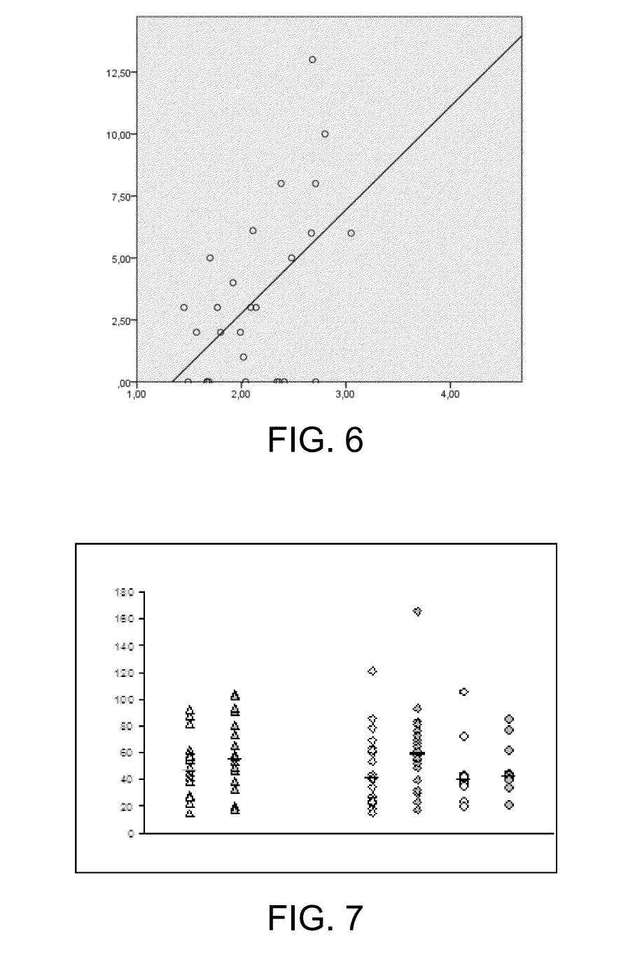

FIG. 6 depicts a comparison of TLs (kb) and deltaMMSE scores (R2=0.284; p=0.008) for total AD patients.

FIG. 7 depicts IL-10 production comparison at day 5 for unstimulated PBMC (open symbols) and Aβ stimulated PBMC (solid symbols).

### DETAILED DESCRIPTION OF THE INVENTION

### Example 1

### Patients and Controls

Forty-seven AD patients and 25 non-demented subjects (HC) were included in a study of Alzheimer's disease. These patients were selected from a larger population sample followed at the Geriatric Department of the Ospedale Maggiore IRCCS, University of Milan, Italy. The DMS IV and NINCDS-ADRDA (23) criteria were adopted to obtain the clinical diagnosis of AD. Cognitive performances and alterations were assessed according to the Mini-Mental State Evaluation (MMSE). AD patients and HC were living at home and were carefully physical examined on the day of blood collection and their clinical records evaluated. In order to minimize the risk of clinical or sub-clinical inflammatory processes, all the patients were selected as follows: only AD and HC without clinical sign of inflammation (e.g. normal body temperature or absence of concomitant inflammatory disease) were included in the study. Blood chemical parameters were also evaluated and subjects with abnormal sedimentation rate of red blood cells or altered blood profile of albumin and transferring plasma levels were excluded. A further selection of AD patients were performed according to the C reactive protein (CRP) plasma levels and those patients with CRP above 5 mg/l (mean value±2 standard deviations of control values) were not enrolled in the study.

Informed consent to perform the study was obtained from controls and a relative of each AD patient.

Blood Sample Collection

Whole blood was collected by venipuncture in Vacutainer tubes containing EDTA (Becton Dickinson Co, Rutherford, N.J.). Peripheral blood mononuclear cells (PBMC) were separated by centrifugation on lymphocyte separation medium (Organon Teknika Corp., Durham, N.C.) and washed twice in PBS. The number of viable lymphocytes was determined by trypan blue exclusion and a hemocytometer.

In Vitro Cytokine Production

PBMCs were resuspended at 3×10⁶/ml in RPMI 1640 and were either unstimulated or stimulated with LPS (Sigma, St. Louis, Mich.) (10 g/ml), with a pool of 3 different peptides from the b-amyloid protein as follows: b-A: fragment 25-35 (25 mg/ml); b-B: fragment 1-40 (150 ng/ml); b-C: fragment 1-16 (150 ng/ml) (Sigma, St. Louis, Mich.); or with influenza virus vaccine (A/Taiwan+A/Shanghai+B/Victoria) (24 g/l; final dilution 1:1000) (Flu) (control antigen) at 37° C. in a moist, 7% CO₂ atmosphere. Supernatants were harvested after 48 hours for LPS stimulation and after 5 days of culture for the b-amyloid protein peptides and Flu. Production of IL-2 and IL-10 by PBMCs was evaluated with commercial available ELISA kits (ACCUCYTE, Cytimmune Sciences, Inc, College Park, Md.). All test kits were used following the procedures suggested by the manufacturer.

IL-10 Genotyping.

Genomic DNA was extracted from EDTA-treated peripheral blood (10 ml) using a standard proteinase K and phenol/chloroform method. The DNA concentration and purity were determined by spectrophotometric analysis. A polymerase chain reaction-sequence specific primers (PCR-SSP) methodology was utilised to assess the IL-10 genotypes. The amplification of the sequence in the promoter region of the IL-10 (polymorphic positions −1082, −819, −592) gene were performed using the Cytokine genotyping Tray Method (One Lambda, Canoga Park, Calif., USA); the human β-globin gene was amplified as an internal control of genomic DNA preparation. PCR condition were indicated by One Lambda PCR program (OLI-1); the PCR products were then visualised by electrophoresis in 2.5% agarose gel.

Statistical Analysis.

Statistical analysis was conducted using SPSS statistical package (SPSS, Chicago, Ill.). Differences in IL-10 production stemmed from analytic procedures based on non parametric analyses (Mann-Whitney); comparisons between different groups of patients were made using Fisher's exact 2-tailed test. Genotype frequencies were compared between the study groups by c² test with an observed significance level of the test below 0.05. Comparisons between the mean values of the age at onset and MMSE in the six different groups of AD were performed by one-way ANOVA analysis.

Age, Gender and MMSE Scores in AD Patients and in HC.

Forty-seven AD patients and 25 age-matched healthy controls were enrolled in the study. The Mini-Mental State Evaluation (MMSE) showed the presence of a mild-to-severe cognitive deterioration in the AD patients. These data are shown in Table I.

MBP-Stimulated IL-10 Production is Reduced in AD Patients.

PBMC of 47 AD patients and of 25 age- and sex-matched HC were stimulated with a mitogen (LPS); a pool of 3 amyloid peptides (A: fragment 25-35, B: fragment 1-40, and C: fragment 1-16) (amyloid), or Flu (used as a control antigen) and the production of IL-2 and IL-10 was measured with ELISA methods. No differences were seen when LPS- or Flu-stimulated IL-2 and IL-10 production was compared in AD patients and HC. Amyloid-stimulated IL-2-production was also similar in the two groups of individuals studied. In contrast with these results, amyloid-stimulated production of IL-10 was significantly reduced (p=0.023) in AD patients compared to controls. These data are shown in FIGS. 1A-1D.

The Distribution of High, Intermediate, and Low IL-10 Producing Genotypes is Skewed in AD Patients.

Paradigmatic example of the six different IL-10 genotypes, evaluated by PCR-SSP, is showed in FIG. 2 and their relative distribution among a typical Caucasian population sample is shown in Table II. In contrast with the distribution observed in HC, the frequency of the different IL-10 genotypes among AD patients was significantly skewed (c²=16.007 with p=0.007) (Table II). Therefore genotypes corresponding to reduced IL-10 production (ACC/ACC, ACC/ATA and ATA/ATA genotypes) had a significantly higher distribution amongst AD subjects (17%, 26% and 11% respectively versus 4%, 16% and 4% in HC). Moreover the GCC/ACC to GCC/ATA ratio (intermediate phenotype) was 1:1 in AD while was 3:1 in HC.

Low IL-10 Production is Correlated with Worsened Clinical Outcome of AD.

To analyse possible clinical correlates of the presence of low IL-10 genotype, we subsequently examined the six genotypes in relation to age of AD onset (Table III) and the progression of cognitive deterioration (Table IV). The results confirmed that the presence of low-IL-10-producing genotypes is indeed associated with a worsened clinical outcome of AD. Thus, presence of the ATA/ATA and GCC/ATA genotypes was associated with an earlier age at disease onset (ANOVA: p=0.042) (Table III); additionally, an inverse correlation was detected between ACC/ATA and ACC/ACC, low IL-10-producing genotypes, and the MMSE score (ANOVA: p=0.010) (Table IV).

TABLE IV
Genetic Association Data for Autoimmune/Inflammatory Disease www.grc.nia.nih.gov/branches/rrb/dna/geneticdata.htm

| Chrom | CH-band | Gene | Disease | Allele | P-value | Reference | PubMedID |
|---|---|---|---|---|---|---|---|
| 1 | 1q31.1 | CD45 | Ms | C to G in position 77 of PTPRC exon 4. | P = 1.510-4 | Jacobsen M 00 | 11101853 |
| 1 | 1q31.1 | CD45 | SCId | deletion | na | Kung C 00 | 10700239 |
| | Mouse | CD45 | autoimmune | glutamate 613 to arginine | na | Majeti R 00 | 11163182 |
| | | | nephritis | | | | |
| 1 | 1q32.1 | IL10 | SLE | −4 kb to 5′ | P = .0001 | Gibson AW 01 | 11238636 |
| 1 | 1q32.1 | IL10 | SS | −10 GCC haplotype (G −1082, C −819, | P = <0.05 | Hulkkonen J 01 | 11212157 |
| | | | | and C −592 of the IL-10 gene | | | |
| 1 | 1q32.1 | IL10 | RA | genotype −1082GG | P = <0.03 | Huizinga TW 00 | 11085795 |
| 1 | 1q32.1 | IL10 | RA | ATA haplotype, pts w/>4 joints | P = 0.02 | Crawley E 99 | 10366102 |
| 1 | 1q32.1 | IL10 | GVHD | IL-10 (−)1064 | P = <.001 | Middleton PG 98 | 9808588 |
| 1 | 1q32.1 | IL10 | IBD/UC | −1082*G allele (high producer) was | P = 0.03 | Tagore A 99 | 10551422 |
| | | | | reduced in pts | | | |
| 2 | 2q12.2 | IL1RA | SLE | IL1RN*2 allele | na | Blakemore AL 94 | 7945503 |
| 2 | 2q12.2 | IL1RA | Ulcerative | IL1RN*2 allele | P = 0.007 | Mansfield JC 94 | 8119534 |
| | | | Colitis | | | | |
| 2 | 2q12.2 | IL1RA | polymyalagia | IL1RN*2 allele | na | Boiardi L 00 | 11138328 |
| | | | rheumatica | | | | |
| 2 | 2q33.1 | CTLA4 | RA | A/G 49 | P = 0.009 | Gonzalez MF 99 | 10203024 |
| 2 | 2q33.1 | CTLA4 | GD | A/G 49 | P = <0.01 | Yanagawa T 97 | 9459626 |
| 2 | 2q33.1 | CTLA4 | MS | A/G 49 | P = 0.006 | Harbo HF 99 | 10082437 |
| 2 | 2q33.1 | CTLA4 | H-Thy | A/G 49 | P = <0.03 | Donner H 97 | 9398726 |
| 2 | 2q33.1 | CTLA4 | IDDM | A/G 49 | P = 0.004 | Takahiro A 99 | |
| 2 | 2q33.1 | CTLA4 | IDDM | | na | Yanagawa T 99 | 10052685 |
| 2 | 2q33.1 | CTLA4 | IDDM | A/G 49 | P = 0.00002 | Marron MP 97 | 9259273 |
| 5 | 5q31.1 | IL4 | GD | position 590 allele reduced in GD | P = 0.00004 | Hunt PJ 00 | 10843185 |
| 5 | 5q31.1 | IL4 | increased IgE | C + 33T polymorphism with elevated | P = <6.05 | Suzuki I 00 | 11122213 |
| | | | | total serum IgE | | | |
| 5 | 5q31.1 | IL4 | asthma, | C-589T IL-4 promoter genotype (TT) | P = 0.013 | Burchard EG 99 | 10471619 |
| | | | FEV(1) | | | | |
| 5 | 5q31.1 | IL4 | AD | −590C/T | P = 0.001 | Kawashima T 98 | 9643293 |
| 5 | 5q31.1 | IL4 | RA | IL-4(2) higher in non-destructive RA | P = 0.0006 | Buchs N 00 | 11035134 |
| 5 | 5q31.1 | IL4 | MS | IL-4 B1 allele, late onset MS | P = <0.001 | Vandenbroeck K 97 | 9184650 |
| 5 | 5q31.1 | IL13 | asthma | Gln110Arg | P = 0.017 | Heinzmann A 00 | 10699178 |
| 5 | 5q31.1 | IL13 | asthma | C to T at position −1055 (TT) | P = 0.002 | van der Pouw | 11197307 |
| | | | | | | Kraan TC 99 | |
| 6 | 6p21.31 | TNFa | asthma | G/A −308 TNF2 | P = 0.003 | Albuquerque R 98 | 9645594 |
| 6 | 6p21.31 | TNFa | PrimBilCirr | G/A −308 TNF1 | P = 0.02 | Gordon M 99 | 10453936 |
| 6 | 6p21.31 | TNFa | Sepsis | G/A −308 TNF2 | P = 0.007 | Majetschak M 99 | 10450735 |
| 6 | 6p21.31 | TNFa | Psoriasis | G/A −308 TNF1 | P = 2.74 × | Arias A 97 | 9395887 |
| | | | | | 10−8 | | |
| 6 | 6p21.31 | TNFa | lep. Leprosy | G/A −308 | P = .02 | Roy S 97 | 9237725 |
| 6 | 6p21.31 | TNFa | GVHD | TNFd | P = .006 | Middleton PG 98 | 9808588 |
| 6 | 6p21.31 | TNFa | Silicosis | G/A −308 TNF1 | P = <0.05 | Yucesoy B 01 | 11264025 |
| 6 | 6p21.31 | TNFa | SLE | G/A −308 TNF1 | na | Sullivan KE 97 | 9416858 |
| 6 | 6p21.31 | TNFa | celiac | G/A −308 TNF1 | P = <0.001 | McManus R 96 | 8655356 |
| 6 | 6p21.31 | TNFa | chronic | G/A −308 TNF1 | P = <0.01 | Huang S 97 | 9372657 |
| | | | bronchitis | | | | |
| 6 | 6p21.31 | TNFa | Psoriasis | −238 TNF1 | P = 1.64 × | Arias A 97 | 9395887 |
| | | | | | 10−7 | | |
| 7 | 7p15.3 | IL6 | IDDM | G,G(−174) increased in pts | P = <0.002 | Jahromi MM 00 | 11054276 |
| 7 | 7p15.3 | IL6 | SLE | AT-rich minisatellite in 3′ flanking | P = 0.001 | Linker-Israeli M 99 | 11197305 |
| | | | | region | | | |
| 7 | 7p15.3 | IL6 | RA | 622 and −174 alleles, age of onset | na | Pascual M 00 | 11196696 |
| 7 | 7p15.3 | IL6 | MS | carriage larger alleles A6-->A9, | P = 0.025 | | |
| | | | | accelerated onset | | | |
| 12 | 12q12 | VDR | GD | exon 2 initiation codon (VDR-FOK:I) | P = 0.023 | Ban Y 00 | 11134121 |
| | | | | polymorphism | | | |
| 12 | 12q12 | VDR | RA | BB/tt genotype | na | Garcia-Lozano JR | 11251690 |
| | | | | | | 01 | |
| 12 | 12q12 | VDR | MS | bb | P = 0.0263 | Fukazawa T 00 | 10465499 |
| 12 | 12q12 | VDR | CD | tt | P = 0.017 | Simmons JD 00 | 10896912 |
| 12 | 12q12 | VDR | IDDM | Bsml | P = 0.015 | Chang T J 00 | 10792336 |
| 12 | 12q21.1 | IFNG | asthma | CA repeat polymorphism within the | P = .0018 | Nakao F 01 | 11240951 |
| | | | | first intron | | | |
| 12 | 12q21.1 | IFNG | IDDM | CA repeat polymorphism within the | P = 0.039 | Awata T 94 | 7867888 |
| | | | | first intron | | | |
| 12 | 12q21.1 | IFNG | GD | CA repeat polymorphism within the | P = <0.04 | Siegmund T 98 | 9848715 |
| | | | | first intron | | | |
| 12 | 12q21.1 | IFNG | RA | CA repeat polymorphism within the | P = <0.0001 | Khani-Hanjani A 00 | 11022930 |
| | | | | first intron | | | |
| 16 | 16p11.1 | IL4R | asthma | Ile50Val | P = <0.0001 | Mitsuyasu H 98 | 9620765 |
| 16 | 16p11.1 | IL4R | hyper-IgE | Arg576G | P = 0.001 | Hershey GKK 97 | 9392697 |
| | | | syndrome and | | | | |
| | | | severe eczema, | | | | |
| | | | atopy | | | | |
| 16 | 16p11.1 | IL4R | MS(PPMS) | IL4R variant R551 | P = 0.001 | Hackstein H 01 | 11164908 |

[DNA Array Unit ]
[IRP Home]
[NIA Home]

### Example 2

### Patients and Controls

Sixty-five AD patients (44 F/21 M, mean age 80+2) and 65 non-demented sex- and age-matched healthy controls (HC) were enrolled. The patients were selected from a larger population sample followed at the Geriatric Department of the Ospedale Maggiore IRCCS, University of Milan, Italy. We applied the DMS IV and NINCDS-ADRDA (23) criteria to obtain the clinical diagnosis of AD; every subject had a recent brain magnetic resonance imaging (MRI)/computed tomography (CT) scan available. Cognitive performances and alterations were assessed according to the Mini-Mental State Evaluation (MMSE). AD patients and HC were living at home and a careful physical examination was done on the day of blood collection, and their clinical records were consulted.

In order to minimize the risk of clinical or sub-clinical inflammatory processes, subjects were selected as follows: only AD and HC without clinical signs of inflammation (e.g. normal body temperature, no concomitant inflammatory condition) were eligible. Blood chemistry tests were done and subjects with an abnormal red blood cell sedimentation rate or altered albumin and transferring plasma levels were excluded. AD patients were further selected according to their C reactive protein (CRP) plasma levels and any with CRP above 5 mg/L (mean+2 standard deviations of control values) were not eligible.

Informed consent was obtained from all the subjects or their relatives. The study protocol was approved by the Ethics Committee of the University Hospital.

Blood Sampling.

Whole blood was collected by venipuncture in Vacutainer tubes containing EDTA (Becton Dickinson Co., Rutherford, N.J.). Peripheral blood mononuclear cells (PBMC) were separated by centrifugation on lymphocyte separation medium (Organon Teknika Corp., Durham, N.C.) and washed twice in PBS. Viable lymphocytes were counted by Trypan blue exclusion and a hemocytometer.

Genotyping.

Genomic DNA was extracted using a standard proteinase K and phenol/chloroform method. The DNA concentration and purity were determined by spectrophotometric analysis. A polymerase chain reaction-sequence-specific primers (PCR-SSP) method was utilised to assess IL-10 and IL-6 genotypes. The sequence in the promoter region of the IL-10 (polymorphic positions −1082, −819, −592) and IL-6 (polymorphic position −174) genes was amplified using the cytokine genotyping tray method (One Lambda, Canoga Park, Calif., USA); the human β-globin gene was amplified as an internal control for the genomic DNA preparation. PCR conditions were indicated by the One Lambda PCR program (OLI-1) and the PCR products were visualised by electrophoresis in 2.5% agarose gel.

The ApoE genotypes were determined by PCR amplification of a 234 base-pair fragment of exon 4 of the ApoE gene, followed by digestion with Cfo1. The restriction patterns were obtained by gel electrophoresis.

In Vitro Cytokine Production.

PBMCs were resuspended at 3×10⁶/mL in RPMI 1640 and were either unstimulated or stimulated with LPS (Sigma, St. Louis, Mich.)(10 μg/mL), with a pool of three peptides from the β-amyloid protein as follows: β-A, fragment 25-35 (25 g/mL); β-B, fragment 1-40 (150 ng/mL); β-C, fragment 1-16 (150 ng/mL) (Sigma), or with influenza virus vaccine (A/Taiwan+A/Shanghai+B/Victoria) (24 μg/L; final dilution 1:1000) (Flu) (control antigen) at 37° C. in a moist, 7% CO₂ atmosphere. Supernatants were harvested after 48 hours for LPS stimulation and after five days of culture for the β-amyloid protein peptides. Production of IL-10 and IL-6 by PBMCs was evaluated with commercial ELISA kits (ACCUCYTE, Cytimmune Sciences Inc., College Park, Md.). All test kits were used following the manufacturer's directions.

Statistical Analysis.

Statistical analysis was done using the SPSS statistical package (SPSS, Chicago, Ill.). Genotype frequencies were compared in the study groups by the χ² test with a level of significance below 0.05. The odds ratio (OR) and 95% confidence intervals (CI) were also calculated. Adjusted ORs were estimated by logistic regression, controlling for ApoE 4 carrier status. Homogeneity of the ORs between strata was assessed by including the appropriate interaction terms in the model. Differences in IL-10 and IL-6 production were established by procedures based on non-parametric analysis (Mann-Whitney); different groups of patients were compared using Fisher's exact two-tailed test.

The Distribution of High, Intermediate, and Low IL-10 Producing Genotypes is Skewed in AD Patients.

The genotype and allele frequencies of the biallelic polymorphism at position −1082 are reported in Table V. This SNP alters transcriptional activation with a gene dosage-related effect, so GG genotype correlates with high, GA with intermediate and AA with low IL-10 production after stimulation of T cells in vitro (57). AD patients show a significantly higher frequency of the −1082A low producer allele, which skews the genotype distribution in AD compared to HC with a significant decrease of −1082GG high producer genotype (Table V).

TABLE V
Frequency of the different IL-10 genotypes and alleles observed in
Alzheimer's disease patients (AD) and in healthy age-matched controls.

| | Genotype | | | Allele | |
|---|---|---|---|---|---|
| | G/G (H)^{a} | G/A (M) | A/A (L) | A | G |
| AD | 4 (6.4%) | 28 (44.4%) | 31 (49.2%) | 90 (71.4%) | 36 |
| | | | | | (28.6%) |
| HC | 14 (22.2%) | 29 (46%) | 20 (31.8%) | 69 (54.8%) | 57 |
| | | | | | (45.2%) |

^{a}The corresponding phenotypes: high (H), intermediate (M), low (L) are shown in brackets
Genotype: χ² = 7.946, df = 2, p = 0.019
Allele: χ² = 6.817, df = 1, p = 0.009

The some SNP is linked with two other SNPs at positions −819 and −592. They combine with microsatellite alleles to form haplotypes where the difference in IL-10 production is mainly accounted for by the −1082 SNP (38, 42). The genotype and allele frequencies of −819 C→T and −592 C→A SNPs were distributed similarly in our AD and HC samples (data not shown).

The −174C Allele in the IL-6 Gene is Over-Represented in AD Patients

The distribution of IL-6 genotypes and alleles in HC and AD is shown in Table VI. This functional polymorphism also seemed related to the plasma IL-6 concentration; however, it is not clear how this SNP influences IL-6 plasma levels (54). The results of the genotype distribution in our AD and HC samples, with a lower frequency of GG genotype in AD patients. Similarly, the allele distribution was significantly different in the two groups, the C allele being significantly higher in AD (Table VI).

TABLE VI
Frequency of the different IL-6 genotypes and alleles observed in
Alzheimer's disease patients (AD) and in healthy age-matched controls.

| | Genotype | | | Allele | |
|---|---|---|---|---|---|
| | G/G (H)^{a} | G/C (H) | C/C (L) | C | G |
| AD | 17 (29%) | 34 (57.6%) | 8 (13.4%) | 50 (42.4%) | 68 (57.6%) |
| HC | 32 (50%) | 27 (42.2%) | 5 (7.8%) | 37 (28.9%) | 91 (71.1%) |

^{a}High (H) and low (L) phenotypes are in brackets
Genotype: χ² = 5.894, df = 2, p = 0.052
Allele: χ² = 4.300, df = 1, p = 0.038

L-10 and IL-6 Allele Combination and Relative Risk of Developing AD

We investigated whether any combination of the IL-10 GA and IL-6 GC alleles affected the risk of AD. The concomitant presence of both IL-10 A and IL-6 C alleles significantly raised this risk, independently of the ApoE4 status (Table VII). The IL-10 NA genotype alone or the IL-6 C/C genotype alone both conferred a smaller increase in the risk of the disease (OR 5.8, CI 1.7-20, p=0.005; OR 3.0, CI 0.9-10.6, p=0.087).

TABLE VII
IL-10 and IL-6 alleles and risk for Alzheimer disease

| IL-10 | IL-6 | OR | 95% CI | adj. OR | 95% CI |
|---|---|---|---|---|---|
| G allele | G allele | 1 | | 1 | |
| G | C | 2.8 | 0.2-40 | 0.9 | 0.1-26.5 |
| A | G | 4.6 | 0.5-41 | 3.3 | 0.3-36.3 |
| A | C | 11.2* | 1.3-97.3 | 10.3* | 1.0-108 |

7* p > 0.05;
OR: crude odds ratio; adj.
OR: apolipoprotein E ε4 adjusted odds ratio;
CI: confidence interval

LPS, Flu, and Amyloid Peptide-Stimulated IL-10 and IL-6 Production is Reduced in AD Patients

PBMC of 47 AD patients and 25 age- and sex-matched HC were stimulated with a mitogen (LPS), with a pool of three β amyloid peptides (βA, fragment 25-35; βB, fragment 1-40; βC, fragment 1-16), or with Flu and the production of IL-10, IL-6 was measured with ELISA methods. There were no differences in LPS- or flu-stimulated IL-6 and IL-10 production in AD and HC. In contrast, when β-amyloid-stimulated production of IL-6 and IL-10 was analysed, a marginal increased IL-6 production and a significant decrement of IL-10 generation (p=0.023) were seen in AD patients compared to HC, suggesting an antigen-specific impairment in the production of these cytokines. These data are shown in FIG. 3.

The causative role of chronic inflammation in the pathogenesis of AD is still mainly speculative (24, 25). Nonetheless a “cytokine cycle” has been proposed where (19) the anti-inflammatory cytokines (IL-4, IL-10 and IL-13) regulate β-amyloid-induced microglial/macrophage inflammatory responses and modify the microglial activity surrounding amyloid neuritic plaques (52). These cytokines can inhibit the induction of IL-1, TNF-α and MCP-1 in differentiated human monocytes and, above all, IL-10 causes dose-dependent inhibition of the IL-6 secretion induced by β-amyloid in these cells and in murine microglia (19).

From a clinical point of view, IL-10 is involved in autoimmune diseases (41, 42, 26) and in malignancies (31, 27, 43) where the higher levels of the cytokine depend on genetic background (59) but also influence the outcome of infections (34, 40, 37).

More consistent is the evidence of a role of IL-6 in the pathogenesis of AD. Elevated IL-6 immunoreactivity was observed close to amyloid plaques in the brain of these patients (67); IL-6 induces the synthesis of β-amyloid precursor protein (69), and in transgenic mouse models elevated CNS levels of IL-6 result in neuropathogenic effects and cognitive deficits (51).

The C allele of a VNTR on the IL-6 gene was reported to reduce cytokine activity (61). The IL-6 VNTR C allele has been correlated with a delayed initial onset and reduced AD risk in a German population (63). The functional polymorphism −178 of the promoter region could also be involved in the development of AD phenotype because of its association with plasma concentrations of the cytokine (54). However, in two clinical sets of different ethnic origin the results were debatable (49).

In our sample the data from SNP analysis showed HC had a distribution of IL-10 and IL-6 alleles similar to that of an Italian population (65). More importantly, the present results point to a significantly higher percentage of IL-10 −1082A carriers among AD cases. A recent report on Italian centenarians, who are clearly less prone than younger persons to age-related diseases, showed that extreme longevity is significantly associated with the high IL-10-producing genotypes (58).

As we have previously reported, the results on IL-6 SNPs are more contradictory. The IL-6 G allele seems significantly in AD of Japanese (66) and also of southern Italian origin (64), whereas in our sample it is the C allele that appears over-represented.

To link these differing findings several points have to be considered. Ethnicity may strongly influence the role of genetic risk factors, and so may the distribution of gene variants in the populations of different European countries, or even among different areas of the same country (53, 55, 60, 62, 70). In addition, the association between AD and IL-6 SNPs may be confined to particular ages, and in our samples AD and HC subjects were all old-old.

Finally, we must consider the role played by a gene or by several genes in linkage disequilibrium with this mutation: a strong disequilibrium between −174 SNP and the VNTR polymorphism of the 3′ flanking region of the IL-6 gene has been described in Germans (49).

The main finding of this study was the identification of a group of subjects with a high risk of late-onset AD on account of the concomitant presence of IL-10 −1082A and IL-6 −174C alleles. We also explored interactions between Apo E and IL-10 or IL-6 genes but did not find any evidence of synergistic effects, suggesting that these inflammation-related alleles are an additional and independent risk factor for AD.

To shed more light on the genetic results, the inventors also analysed β-amyloid peptide-, LPS-, and Flu-specific IL-10 and IL-6 production by peripheral blood mononuclear cells (PBMC) in a subset of AD patients and age-matched

HC. The results showed that: 1) IL-6 production by PBMC of AD patients and controls did not differ significantly in any conditions; and 2) IL-10 generation by LPS- and Flu-stimulated PBMC was comparable in the two groups, whereas a β-amyloid-specific immune impairment characterized by a reduced generation of IL-10 was noted in AD. The fact that this cytokine imbalance was not seen in mitogen-stimulated PBMC indicates that β-amyloid-specific immune responses are selectively impaired in AD. Additionally, the finding that flu-stimulated proliferation was similar in patients and controls indicates that antigenic processing and presentation in association with HLA class II molecules, and the CD4-HLA class II self-restricted pathway of activation of the immune system (44), are not defective in AD. Thus a biological scenario is conceivable in which the reduction of amyloid-specific IL-10 production favours the triggering of the chronic inflammatory process seen in the AD brain. An amyloid-specific and IL-10-mediated inhibitory feedback circuit could be active in non-AD individuals, and a breakdown of this circuit could be associated with, or predictive of, the development of AD. A recent study showed convincingly that an IL-10/pro-inflammatory circuit revolving around cells of the innate immune system regulates susceptibility to autoimmune diseases (48). Our results extend this concept by showing that in AD patients this circuit is altered. The data as a whole support the theory that the overall risk of developing AD may be governed by a “susceptibility profile”, that reflects the combined influence of inheriting multiple high-risk alleles, and casts light on the pivotal role of IL-10 and IL-6 SNPs in this profile.

Inflammation is involved in the pathogenesis of Alzheimer's disease (AD, the anti-inflammatory cytokine interleukin-10 (IL-10) might counteract IL-6 activity in the brain. As the promoter of these genes is polymorphic, the 65 AD patients and 65 healthy controls (HC) the present investigated the IL-10 −1082 GA and IL-6 −174 GC alleles. In several cases they also assessed IL-10 and IL-6 production by PBMC. For IL-10 there was a significant higher level of the −1082GG genotype (p=0.019) in HD than HC, while for IL-6 the G/G genotype was lower and the C allele higher (p<0.005). The concomitance of IL-10 A and IL-6 C alleles significantly raised the risk of AD (odds ratio: OR 11.2, confidence interval: CI 1.3-97.3; p<0.05) independently of ApoE4 (adjusted OR 10.3, CI 1-108; p<0.05). Only amyloid-stimulated IL-10 production differed in AD and HC (p=0.023). These results conflict with the inflammatory theory in AD, pointing to a pivotal role of IL-10 and IL-6 polymorphisms and a selective alternation in this network.

### Example 3

It is commonly accepted that Alzheimer's disease (AD) pathology starts years to decades before the onset of cognitive symptoms (78). This fact explains why symptomatic AD consistently represents an advanced stage of AD pathology (79).

Amyloid plaques and neurofibrillary tangles are the pathological hallmarks of AD. However, considering AD as a single, well-defined entity has become an obstacle to lucid analysis of the problem of dementia in old age. Whereas in early-onset patients AD might be best designated as a purely degenerative disease with an important role for amyloid-beta in the pathogenesis, in late-onset patients (LOAD) the probability of finding other abnormalities is increased (80-82). To date, 90% of all AD patients in the population are older than 75 years, and 75% of patients are over 80 years of age (83). This support that aging and pathophysiological changes it induces may be concauses of AD onset and progression in elderly and raise the need to find novel biomarkers.

Telomeres are specialized sequences consisting of highly conserved TTAGGG repeats (84-86) that cap the ends of linear chromosomal DNA, protecting the genome from damage and preserving chromosome stability (85, 87-89). However, because of their end positions, telomeres are not fully duplicated during DNA replication and thus become shorter with each cell division (99-92). This process limits the replicative lifespan of many different cells that, eventually, enter a senescent status or trigger apoptosis (93-98). Telomere length (TL) reflects not only cellular turnover but also the exposure to oxidative and inflammatory damage (99-104) and, accordingly, may be a marker of both biological age and mortality risk (102, 105-107) that predicts incidence of age-related diseases (108-112).

To date, investigations on blood cells have been inconsistent with respect to the relationship between telomere shortening and AD (113-122). In particular, several studies reported TL is associated with cognitive decline in elders (123-126) and is reduced in AD patients (113-119) but other studies showed TL is not associated with either levels of cognitive performance or age-related cognitive change (127, 128) and cannot be used in elderly as marker to distinguish between demented and non-demented patients, regardless of the type of dementia, or to predict dementia (120-122).

A growing body of literature shows that inflammation is involved in the neurodegeneration process (129-133) and can furthermore accelerate telomere shortening (101-104) which, in turn, may be linked with the pathogenesis of AD (113-119, 134-136). Interestingly, it is suggested that anti-inflammatory cytokines, like interleukin-10 (IL-10), may partly antagonize this processes (137, 138). In particular, IL-10 has been suggested to play an important role in neuronal homeostasis and may be also able of inhibiting β-amyloid (Aβ) or LPS induced generation of proinflammatory cytokynes (139). Our previous case-control investigation showed a decreased Aβ stimulated production of IL-10 in AD patients, suggesting an antigen-specific impairment in the production of this cytokine (140).

Materials and Methods.

Patients and Controls.

A total of 61 individuals were enrolled in the study: 20 healthy elderly (HE) (mean age 79.1±8.4); 30 LOAD patients (mean age 80.4±5.1). Subjects diagnosed with AD fulfilled the criteria of dementia and the criteria of AD defined by NINCDS-ADRDA (64). All the individuals were Caucasians living in Northern Italy or France and belonged to larger populations of outpatients attending the two units.

The criteria for the diagnosis of normal cognition were: (1) no active neurological or psychiatric disorder; (2) no ongoing medical problems or related treatments interfering with cognitive function; (3) a normal neurological exam; (4) no psychoactive medications, and (5) the ability to live and function independently in the community.

Individuals affected by cancer or cardiovascular disease were not included in the study and, at recruitment.

All participants and their relatives gave informed consent and the study protocol was approved by the respective university hospital's ethics committees.

At baseline, physical, neurological, and neuropsychological examinations were performed for all LOAD patients together with clinical history, computer tomography or magnetic resonance imaging scan and cognitive testing using mini-mental state examination (MMSE). Laboratory analyses included apolipoprotein E (ApoE) genotype assessment and biochemical tests.

After a two-year period of follow-up LOAD patients were retrospectively evaluated and AD progression rate was assessed by deltaMMSE score (MMSE score at recruitment—MMSE score after the follow-up period). Based on deltaMMSE score, patients were categorized as slow-progressing AD (ADS) if deltaMMSE≦4 points or fast-progressing AD (ADF) if deltaMMSE≧5 points (142).

Blood from all individuals was collected at the same time in the morning after the follow-up period.

ApoE Genotyping.

Genomic DNA was extracted from whole blood by using a salting-out method (66) and ApoE genotype was determined as previously described (140).

PBMC Isolation.

PBMC were isolated from whole blood by density gradient using the Lympholyte-H kit (Cedarlane Laboratories Limited, Burlington, ON) and stored with 10% DMSO at −80° C. pending analysis.

Flow Cytometry.

TL was determined by flow cytometry using the telomere PNA kit/FITC® (Dako Italia, Milan, IT) following manufacturer's instructions. This Kit allows calculating the relative telomere length (RTL) of sample cells (SC) using control cells (CC) with a known telomere length. The tetraploid 1301 cell line (Biologic Bank and Cell Factory, Genoa, IT) with a TL of approximately 25 Kb (144) was used as CC. A 1:1 mixture of SC and CC were prepared and 2 positive and 2 negative samples were included in each evaluation. In all samples, DNA was denatured for 10 minutes at 82° C. either in the presence of hybridization solution without probe (negative) or in hybridization solution containing fluorescein-conjugated PNA telomere probe (positive). Hybridization was carried out overnight, in the dark at room temperature. After two washes at 40° C., samples were stained with propidium iodide (PI) for 4 hours at 4° C. for the identification of cells in G0/G1 phases and the DNA index calculation. Samples were then acquired with a FACSCanto II (Becton Dickinson, Franklin Lakes, N.J.) and analyzed with a Kaluza® software (Beckman Coulter, Indianapolis, Ind.).

RTL was calculated with the following formula with correction for the DNA index of G0/1 cells:

$$RTL = \frac{\begin{matrix}( \begin{matrix}mean FL 1 sample cells with probe - \\ mean FL 1 sample cells without probe\end{matrix} ) \times \\ DNA index of control cells \times 100\end{matrix}}{\begin{matrix}( \begin{matrix}mean FL 1 control cells with probe - \\ mean FL 1 control cells without probe\end{matrix} ) \times \\ DNA index of sample cells\end{matrix}}$$

The absolute TL was calculated multiplying RTL for 25 Kb.

Telomere Polimerase Chain Reaction.

Genomic DNA was extracted from PBMC (143) and stored at −80° C. pending analysis. To confirm flow cytometry data, TL was reassessed by using a modified quantitative PCR (q-PCR) Cawthon method (141). We determined the relative ratio (T/S ratio) of telomere (T) repeat copy number to a single copy gene (S) copy number using a comparative quantitation approach. Primer pairs, their final concentration and the thermal cycling profiles were exactly as described (145) except that the number of amplification cycles was increased to 30 and 40 for the T and S reactions respectively. An aliquot of 10 ng (10 μl) template DNA was added containing 12 μl SYBR Select Master Mix (Applied Biosystem, Foster City, Calif.). The final volume of each reaction was 25 μl. Before running samples, the linear range of T and S assay was determined by generating a standard curve using a serially diluted DNA (from 70 to 2.2 ng in 2-fold dilutions). Both T and S reactions showed good linearity across this input range (r²>0.99). On each plate a calibrator sample (a mixture of several DNAs) and a negative control were included. Each sample, including the calibrator, was run in triplicate. All q-PCR assays were performed on an ABI 7500 system (Applied Biosystem, Foster City, Calif.). For each T and S q-PCR assay, raw data were exported from the ABI system and imported into the LinRegPCR program (69) which automatically determined the fluorescence threshold for all samples and calculated the individual threshold cycle and the mean efficiency of the run. Mean efficiency was used in calculating the T and S relative concentration of each sample relative to the calibrator sample (146). TL was expressed as T/S ratio. The inter-assay coefficient of variation (CV) for TL measurement was <5%.

Medium, LPS or Aβ Stimulated IL-10 Production.

Statistical Analysis.

Statistical analysis was performed by using SPSS statistical package (SPSS version 20, Chicago, Ill.). Sex and APOE ε4 allelic distribution in the study groups were assessed by the χ2-test. Demographic data, cognitive performance and TL were examinated by ANOVA univariate, followed by Bonferroni post-hoc test. The correlation between TLs measured by flow cytometry (kb) and TLs measured by q-PCR (T/S ratio) and between TLs (kb) and deltaMMSE scores was performed by linear regression analysis. Differences in IL-10 levels between groups were evaluated by non-parametric analysis (Mann-Whitney test) while the difference, within groups, in IL-10 production between Aβ stimulated and unstimulated PBMC was assessed by ANOVA for repeated measures. P<0.05 was used as the threshold value for the statistical significance.

Results.

TABLE VIII
Demographic and clinical characteristics of slow-progressing AD
patients (ADS), fast-progressing AD patients (ADF)
and healthy elderly individuals (HE)

| | ADS | ADF | HE | p |
|---|---|---|---|---|
| | (n = 20) | (n = 10) | (n = 20) | value |
| Age, mean ± S.D. | 80.8 ± 5.7 | 79.8 ± 3.9 | 79.1 ± 8.4 | N.S. |
| Gender, female, % | 60 | 80 | 50 | N.S. |
| APOE ε4, non | 44 | 40 | 75^{a} | <0.05 |
| carriers, % | | | | |
| MMSE at | 20.6 ± 4.4 | 20.5 ± 5.4 | N.V. | N.S. |
| recruitment, | | | | |
| mean ± S.D. | | | | |
| MMSE after the | 19.4 ± 4.5^{b} | 13.0 ± 5.1^{b,c} | 28.9 ± 1.5 | <0.001 |
| follow-up, | | | | |
| mean ± S.D. | | | | |
| deltaMMSE, | 1.4 ± 1.4 | 7.4 ± 2.8^{c} | NV | <0.001 |
| mean ± S.D. | | | | |
| TL (flow | 2.0 ± 0.4 | 2.5 ± 0.4^{d} | 2.3 ± 0.4^{e} | <0.001 |
| cytometry), kb, | | | | |
| mean ± S.D. | | | | |

^{a}p<? vs all LOAD patients (ADS + ADF);
^{b}p < 0.001 vs HE;
^{c}p < 0.001 vs ADS;
^{d}p = 0.003 vs ADS;
^{e}p = 0.022 vs ADS

Characteristic of Study Population.

Table VIII displays the demographic and clinical characteristics of participants. No difference in age or gender distribution was observed between HE, ADS and ADF. In agreement with literature data, AD patients showed an higher frequency of APOE ε4 allele compared to HE (p<0.05) (147, 148). At recruitment, ADS and ADF showed no difference in mean MMSE score while, after the follow-up period, ADF exhibited a lower score (p<0.001). Accordingly, deltaMMSE, indicating the rate of disease progression, was significantly higher in ADF (p<0.001).

Telomere Length Analysis.

A comparison was made of TL (mean±S.D.) between LOAD patients and HE; no difference was found (2.2±0.5 vs 2.3±0.4 Kb respectively). FIG. 4A depicts telomere length for HE and total AD.

Subsequently, patients were categorized as fast or slow progressing based on disease progression rate. This further classification led to the finding that ADS displayed shorter telomeres not only compared to HE (2.0±0.4 vs 2.3±0.4 kb; p=0.022) but also to ADF (2.0±0.4 vs 2.5±0.4 kb; p=0.003) as seen in Table VIII and FIG. 4B. FIG. 4B depicts telomere length between HE, ADS and ADF, where *p=0.022 vs. ADS and **p=0.003 vs. ADS.

Flow cytometry TLs (kb) were compared to q-PCR TLs (T/S) and a very strong correlation was found (R2=0.523; p<0.001) as seen in FIG. 5. In FIG. 5, the correlation was adjusted for sex, age and APOE ε4 genotype between TLs assessed by flow cytometry (kb) and TLs measured by quantitative PCR (T/S) (R²=0.523, p<0.001).

To confirm the progressive telomere shortening with aging, TL was also measured in HY who showed longer telomeres compared to the other groups (3.2±0.5 kb; p<0.001).

TL Directly Correlates with Delta MMSE Score.

A direct correlation was found in total AD patients between TLs (kb) and deltaMMSE scores (R2=0.284; p=0.008), suggesting a possible role for TL as predictive marker of AD progression rate as shown in FIG. 6. In FIG. 6, the correlation was adjusted for sex, age and APOE ε4 genotype between TL and delta MMSE score in AD patients (R2=0.284; p=0.008).

IL-10 Production in Resting and Aβ or LPS Stimulated PBMC.

TABLE IX
IL-10 production from unstimulated PBMC and after stimulation with
LPS or β-amyloid

| | ADS | ADF | HE | p |
|---|---|---|---|---|
| | (n = 20) | (n = 10) | (n = 18) | value |
| Unstimulated IL-10 | 23.2 ± 5.7 | 16.2 ± 4.9 | 21.0 ± 6.0 | N.S. |
| production (day 2), | | | | |
| median ± M.A.D. | | | | |
| Unstimulated IL-10 | 40.7 ± 13.7 | 39.7 ± 14.4 | 47.1 ± 25.4 | N.S. |
| production (day 5), | | | | |
| median ± M.A.D. | | | | |
| LPS-stimulated IL-10 | 671 ± 285 | 797 ± 223 | 485 ± 249 | N.S. |
| production (day 2), | | | | |
| median ± M.A.D. | | | | |
| Aβ-stimulated IL-10 | 59.0 ± 27.0 | 42.2 ± 22.4 | 55.3 ± 27.9 | N.S. |
| production (day 5), | | | | |
| median ± M.A.D. | | | | |

PBMC from 20 ADS, 10 ADF and 18 age-and-sex matched HE were stimulated with a pool of three Aβ peptides (fragment 1-16; fragment 25-35; fragment 1-40), or a mitogen (LPS) and incubated for the indicated times before. The production of IL-10 was measured by ELISA assay after stimulation and in resting PBMC.

There was no difference in IL-10 production between the study groups (Table IX). In contrast, when Aβ stimulated and unstimulated IL-10 production at day 5 were compared within groups, a significant positive difference was found in both HE (55.3±27.9 vs 47.1±25.4; p=0.05) and ADS (59.0±27.0 vs 40.7±13.7; p=0.005). Interestingly, ADF did not show such a difference, suggesting a lack of response to Aβ stimulus (42.2±22.4 vs 39.7±14.4; p=0.671) as seen in FIG. 7. In FIG. 7, IL-10 production comparison at day 5 is depicted for unstimulated PBMC (open symbols) and Aβ stimulated PBMC (solid symbols).

### CONCLUSIONS

Even though telomere shortening has been associated with neurodegeneration, dementia risk and AD etiopathogenesis, literature data have shown conflicting results. Our study showed no difference in TL between AD subjects compared to controls. Neverthless, when patients were categorized based on disease progression rate, a difference in TL was found. In line with our results, a recent in vivo study showed that telomere shortening is associated with a slower cognitive decline in APP23 mice, a mouse model of AD (151).

Inflammation is considered to be involved in AD etiopathogenesis through effects on neuronal omeostasis and immune response (129-133). In addiction, the immune system of AD subjects is reported to be poorly responsive to Aβ as it exhibits a reduced ability to phagocytize amyloid peptides (149) and a severe lack of proliferative responsiveness to amyloid stimulus (150). Our previous study showed a decreased Aβ stimulated production of IL-10 in PBMC from AD patients (140) and the present data highlighted in ADF a lack of IL-10 increase after Aβ stimulus. We speculate that PBMC from ADF may be characterized by an impaired capacity to respond to inflammatory stimulus induced by Aβ. This may lead to a reduced proliferative response that, in turn, may be responsible for the longer telomeres in ADF.

Our data and APP23 mice investigations (151) showed shorter telomeres in those exhibiting a slower rate of disease progression. However, considering telomere shortening as a beneficial process with respect to the rapidity of AD aggravation requires further experimental and clinical evidences.

The present study's strength is the further classification of AD patients in two sub-groups, which are characterized by a different progression rate (and may display different peculiar characteristics). Weaknesses include: (1) the small size of the study population. This may be responsible for the absence of significant differences in IL-10 productions between groups, notwithstanding the disparity in terms of median values between ADS and ADF (Table IX); (2) the lack of available cells from the enrolled subjects for the Aβ-stimulated proliferation activity analysis.

The data suggest that PBMC as peripheral biomarkers that may mirror alterations within the diseased brain. Moreover, we show a longer TL and a lack of IL-10 increase in response to Aβ stimulus in ADF. The significant direct correlation between TLs and delta MMSE scores indicates TL may have a role as predictive marker of AD progression rate. The assessment of how rapidly AD is aggravating has important implications in clinical practice, since the rate of disease progression may be the most important factor in determining prognosis (152, 153).

Other investigations, with larger study groups, are necessary to clarify the mechanisms responsible for the different course of the pathology in ADS and ADF.

To further explore the contribution of telomere shortening to the onset and progression rate of AD, we measured TL in peripheral blood mononuclear cells (PBMC) from LOAD patients and age-matched controls and we correlated it with disease progression rate. We also assessed, in most of these individuals, whether TL was associated with IL-10 production by resting and Aβ or LPS stimulated PBMC. Here we report that TL is directly associated with the rate of cognitive decline during the follow-up period and may thus be considered as a predictive marker of AD progression rate. In addiction an impaired response to Aβ stimulus may contribute to cause a faster AD progression.

### REFERENCES

1. Ernst, R. L. and J. W. Hay. 1994. The US economic and social costs of Alzheimer's disease revised. Am. J. Public Health. 84: 1261.
2. Goate, A., M. C. Chartier-Harlin, M. Mullan, J. Brown, F. Crawford, L. Fidani, L. Giuffra, A. Haynes, N. Irving, L. James, R. Mant, P. Newton, K. Rook, P. Rogues, C. Talbot, M. Pericak-Vance, A. Roses, R. Williamson, M. Rossor, M. Owen and J. Hardy. 1991. Segregation of a missense mutation in the amyloid precursor protein gene with familial Alzheimer's disease. Nature. 349: 704.
3. Levy-Lahad, E., W. Wasco, P. Poorkaj, D. M. Romano, J. Oshima, W. H. Pettingell, C. E. Yu, P. D. Jondro, S. D. Schmidt, K. Wang, A. C. Crowley, Y. H. Fu, S. Y. Guenette, D. Galas, E. Nemens, E. M. Wiisman, T. D. Bird, G. D. Schellemberg and R. E. Tanzi. 1995. Candidate gene for the chromosome 1 familial Alzheimer's disease locus. Science. 269: 973.
4. Sherrington, R., E. I. Rogaev, Y. Liang, E. A. Rogaeva, G. Levesque, M. Uveda, H. Chi, C. Lin, G. Li, K. Holman, T. Tsuda, L. Mar, J. F. Fonci, A. C. Bruni, M. P. Montesi, S. sorbi, I. Rainero, L. Pinessi, L. Nee, I. Chumaken, D. Pollen, A. Brookes, P. Sanseau, R. J. Polinsky, W. Wasco, H. A. R. Da Silva, J. L. Haines, M. A. Pericak-Vance R. E. Tanzi, A. D. Roses, P. E. Fraser, J. M. Rommens and P. H. George-Hyslop. 1995. Cloning of a gene bearing missense mutation in early-onset familial Alzheimer's disease. Nature. 375: 754.
5. Blacker, D., J. L. Haines, L. Rodes, H. Terwedow, R. C. P. Go, L. E. Harrel, R. T. Perry, S. S. Basset, G. Chase, D. Meyers, M. S. Albert and R. Tanzi. 1997. ApoE-4 and age at onset of Alzheimer's disease: The NINH Genetics Initiative. Neurology. 48: 139.
6. Poirier, J., J. Davignon, D. Bouthillier, S. Kogan, p. Bertrand and S. Gauthier. 1993. Apolipoprotein E polymorphism and Alzheimer's disease. Lancet. 342: 697.
7. Saunders, A. M., W. J. Strittmatter, D. Schmechel, P. H. George-Hyslop, M. A. Pericak-Vance, S. H. Joo, B. L. Rosi, J. F. Gusella, D. R. Crapper-MacLachlan and M. J. Alberts. 1993. Association of apolipoprotein E allele epsilon 4 with late-onset familial and sporadic Alzheimer's disease. Neurology. 43:1467.
8. Fassbender, K., C. Masters and K. Beyreuther. 2000. Alzheimer's disease: an inflammatory disease?. Neurobiology of Aging. 21: 433.
9. McGeer, P. L. and E. G. McGeer. 2001. Inflammation, autotoxicity and Alzheimer disease. Neurobiology of Aging. 22: 799.
10. Zandi, P. P and J. C. S. Breitner. 2001. Do NSAIDs prevent Alzheimer's disease? And, if so, why? The epidemiological evidence. Neurobiology of Aging. 22: 811.
11. Rogers, J., L. C: Kirby, S. R. Hempelman, D. L. Berry, P. L. McGeer, A. W. KasniaK, j. Zalinski, M. Cofield, L. Mansukhani, and P. Willson. 1993. Clinical trial of indomethacin in Alzheimer's disease. Neurology. 43: 1609.
12. Hauss-Wegrzyniak, B., L. B. Willard, P. Del Soldato, G. Pepeu and G. L. Wenk. 1999. Peripheral administration of novel anti-inflammatories can attenuate the effects of chronic inflammation within the CNS. Brain Res. 815: 36.
13. Neuroinflammation Working Group. 2000. Inflammation and Alzheimer's disease. Neurobiology of Aging. 21: 383.
14. Licastro, F., S. Pedrini. L. Caputo, G. Annoni, L. J. Davis, C. Ferri, V. Casadei and L. M. E. Grimaldi. 2000. Increased plasma levels of interleukin-1, interleukin-6 and a-1-antichymotrypsin in patients with Alzheimer's disease: peripheral inflammation or signals from brain?. Journal of Neuroimmunology. 103: 97.
15. Mrak, R. E. and W. S. T. Griffin. 2001. Interleukin-1, neuroinflammtion, and Alzheimer's disease. Neurobiology of Aging. 22: 903.
16. Perry, R. T., J. S. Collins, H. Wiener, R. Acton and R. C. P. Go. 2001. The role of TNF and its receptors in Alzheimer's disease. Neurobiology of Aging. 22: 873.
17. Wyss-Coray, T., E. Masliah, M. Mallory, L. McConlogue, K. Johnson-Wood, C. Lin and L. Mucke. 1997. Amyloidogenic role of cytokine TGF-beta1 in trangenic mice and in Alzheimer's disease. Nature. 389: 603.
18. Meda, L., P. Baron, E. Prat, E. Scarpini, R. Delgado, A. Catania, J. M. Lipton and G. Scarlato. 1999. Proinflammatory profile of cytokine production by human monocytes and murine microglia stimulated with beta-amyloid. J. Immunol. 93: 45.
19. Szczepanik, A. M., S. Funes, W. Petko and G. E. Ringheim. 2001. II-4, IL-10 and II-13 modulate A beta (1-42)-induced cytokine and chemokine production in primary murine microglia and a human monocyte cell line. J. Neuroimmunol. 113: 49.
20. Lombardi, V. R., M. Garcia, L. Rey and R. Cacabelos. 1999. Characterization of cytokine production, screening of lymphocyte subset patterns and in vitro apoptosis in healthy and Alzheimer's Disease (AD). J. Immunol. 97: 163.
21. Engelborghs, S., M. De Brabander, J. De Cree, R. D'Hooge, H. Geerts, H. Verhaegen and P. P. De Deyn. 1999. Unchanged levels of interleukins, neopterin, interferon-gamma and tumour necrosis factor-alpha in cerebrospinal fluid of patients with dementia of the Alzheimer type. 34: 523.
22. Hutchinson, I. V., V. Pravica and P. J. Sinnot. 1998. Genetic regulation of cytokine synthesis: consequences or acute and chronic organ allograft rejection. Graft. 1: 15.
23. McKhann, G., D. Drachman, M. Folstein, R. Katzman, D. Proce, E. M. Stadlan. 1984. Clinical diagnosis of Alzheimer's disease. Neurology. 34: 939.
24. Eikelenboom, P., S. S. Zhan, W. A. van Goll and D. Allsop D. 1994. Inflammatory mechanisms in Alzheimer disease. Trends Pharmacol Sci 15:447.
25. Rogers, J., S. Webster, and L. F. Lue. 1996. Inflammation and Alzheimer's disease pathogenesis. Neurobiol Aging 17:686.
26. Llorente, L., W. Zou, Y. Levy, Y. Richaud-Patin, Y. Wijdenes, J. Alcocer-Varela, B. Morel-Fourrier, J. C. Brouet, D. Alarcon-Segovia, P. Galanaud. 1995. Role of interleukin 10 in the B lymphocyte hyperactivity and autoantibody production of human systemic lupus erythemattosus. J. Exp. Med. 181: 839.
27. Luscher, U., L. Filgueira, A. Juretic, M. Zuber, L. J. Luuscher, M. Heberer and g. C. Spagnoli. 1994. The pattern of cytokine gene expression in freshly excised human metastatic melanoma suggests a state of reversible anergy of tumor-infiltrating lymphocytes. Int. J. Cancer. 57: 612.
28. Matsuda, M., F. Slazar, M. Petersson, G. Masucci, J. Hansson, Q. C. Zhang, M. G. Masucci and R. Kiessling. 1994. Interleukin 10 pretreatment protects target cells from tumor- and allo-specific cytottoxic T cells and downregulates HLA class I expression. J. Exp. Med. 0.180:
29. Kim, J., R. L. Modlin, R. L. Moy, S. M. Dubinett, T. McHugh, B. J. Nickloff and K. Uyemura. 1995. IL-10 production in cutaneous basal and squamous cell carcinomas. A mechanism for evading the local T cell immune response. J. Immunol. 155: 2240.
30. Suzuki, T., H. Tahara, S, Narula, K. W. Moore, P. D. Robbins, and M. T. Lotze. 1995. Viral interleukin 10 (IL-10), the human herpes virus 4 cellular IL-10 homologue, induces local anergy to allogenic and syngenic tumors. J. Exp. Med. 182: 447.
31. Fortis, C., M. Foppoli, L. Gianotti, L. Galli, G. Citterio, G. Consogno, O. Gentilini and M. Braga. 1196. Increased interleukin-10 serum levels in patients with solid tumours. Cancer Lett. 104: 1.
32. Murray, P. J., L. Wang, R. C. Onufry, R. I. Tepper, and R. A. Young. 1997. T cell-derived IL-10 antagononizes macrophage function in mycobacterial infection. J. Immunol. 158: 315.
33. Lehmann, A. K., A. Halstenen, S. Sornes, O. Rokkeand A. Waage. 1995. High levels of interleukin 10 in serum are associated with fatality in meningococcal disease. Infect. Immun. 63: 2109.
34. Clerici, M., T. A. Wynn, J. A. Berzofsky, R. L. Coffman, A. Sher, G. M. Shearer. 1994. Role of Interleukin-10 (IL-10) in T Helper Cell Dysfunction in Asymptomatic Individuals Infected with the Human Immunodeficiency Virus (HIV-1). J. Clin. Invest. 93:768.
35. VanFurth, A. M., E. M. Seijmonsbergen, J. A. M. Langermans, P. H. P. Groeneveld, C. E. Debel and R. VanFurth. 1995. High levels of interleukin 10 and tumor necrosis factor alpha in cerebrospinal fluid during the onset of bacterial meningite. Clin. Infect. Dis. 21: 220.
36. Llorente, L., Y. Richaud-Patin, J. Couderc, D. Alarcon-Segovia, R. Ruiz-Soto, N. Alcocer-Castillejos, J. Alcocer-Varela, J. Granados, S. Bahena, P. Galanaud and D. Emilia. 1997. Dysregulation of interleukin-10 production in relatives of patients with systemic lupus erythematosus. Arthritis Rheum. 38: 1429.
37. Westendorp, R. G. J., J. A. M. Langermans, T. W. G. Huizing a, A. H. Elouali, C. L. Verwej and J. P. Vandenbroucke. 1997. Genetic influence on cytokine production and fatal meningococcus disease. Lancet. 349:170.
38. Eskdale, J., G. gallagher, C. L. Vermeij, V. Keijsers, R. G. J. Westendorp and T. W. J. Huizing a. 1998. Interleukin 10 secretion in relation to human IL-10 locus haplotypes. Proc. Natl. Acad. Sci. USA. 95: 9465-9470.
39. Derkx, B., A. marchant, M. Goldman, R. Biilmer and S. Van de Venter. 1995. High levels of interleukin-10 during the initial phase of fulminant meningococcal septic shock. J. Infect. Dis. 171: 229.
40. Llorente, L., Y. Richaud-Patin, R. Fior, J. Alcocer-Varela, J. Wijdnes, B. Morel-Fourrier, P. Galanaud and P. Emilie. 1994. In vivo production of interleukin-10 by non-T cells in rheumatoid arthritis, Sjogren's syndrome, and systemic lupus erythematosus. A potential mechanism of B lymphocytes hyperactivity and autoimmunity. Arthritis Rheum. 37: 1647.
41. Cash, J. J., J. B. Splawski, R. Thomas, J. F. McFarlin, H. Schulze-Koops, L. S. Davis, K. Fujita and P. E. Lipsky. 1995. Elevated interleukin-10 levels in patients with rheumatoid arthritis. Arthritis Rheum. 38: 96.
42. Eskdale, J. P. Wordsworth, S. Bowman, M. Field and G. Gallagher. 1997. Association between polymorphisms at the human IL-10 locus and systemic lupus erythematosus. Tissue Antigens. 49: 635.
43. Zheng, C., D. Huang, L. Liu, R. Wu, S. Bergenbrant Glas, A. Ostenborg, M. Bjorkholm, G. Holm, Q. Yi, A. Sundblad. 2001. Interleukin-10 gene promoter polymorphisms in multiple myeloma. Int. J. Cancer. 95: 184.
44. Via, C. S., G. C. Tsokos, N. I. Stocks, M. Clerici and G. M. Shearer. 1990. Human in vitro allogeneic responses: demonstration of three pathways of T helper cell activation. J. Immunol. 144:2524.
45. Hahn, A. B., J. C. kasten-Jolly, D. M. Costantino, E. Graffunder, T. P. Singh, G. K. Shen and D. J. Conti. 2001. Tnf-a, II-6, IFN-g, and IL-10 gene expression polymorphisms and the IL-4 receptor a-chain variant Q576R: effects on renal allograft outcome. Transplantation. 72: 660.
46. Lio, D., G. Candore, A. Colombo, G. Colonna Romano, F. Gervasi, V. Marino, L. Scola and C. Caruso. 2001. A genetically determined high setting of TNF-αlpha influences immunologic parameters of HLA-B8, DR3 positive subjects: implications for autoimmunity. Hum Immunol. 62: 705.
47. Akdis, C. A., and K. Blaser K. 2001. Mechanisms of interleukin-10-mediated immune suppression. Immunol. 103:131.
48. Segal, B. M., B. K. Dwyer and E. M. Shevach. 1998. An interleukin (IL)-10/IL-12 immunoregulatory circuit controls susceptibility to autoimmune disease J Exp Med 187: 537.
49. Bagli M, Papassotiropuolos A, Knapp M, Jessen F, Rao M L, Maier W, Heun R. association between an interleukin-6 and 3′ flanking region haplotype and reduced Alzheimer's risk in German population. Neurosci Lett 2000; 283: 109-112.
50. Bowcock A M, Kidd J R, Lathrop G M, Daneshvar L, May L T, ray A, Sehgal P B, Kidd K K, Cvalli-Sforza L L. The human “interferon-beta 2/hepatocyte stimulating factor/IL-6” gene: DNA polymorphism studies and localization to chromosome 7p21. Genomics 1988; 3(1): 8-16.
51. Campbell I L, Stalder A K, Chiang C S, Bellinger R, Heyser C J, Steffensen S, Masliah E, Powell H C, Gold L H, henriksen S J, Siggins G R. Transgenic models to assess the pathogenic actions of cytokines in the central nervous system. Mol Psychiatry 1997; 2: 125-129.
52. Chao C C, Molitor T W, Hu S, Neuroprotective role of IL-4 against activated microglia. J Immunol 1993; 151: 1473-1481.
53. Falcone E, Spadafora P, De Luca M, Ruffolo R, Brancati C, De Benedictis G. DYS19, D12S67, and D1S80 polymorphisms in population sample from southern Italy and Greece. Hum Biol 1995; 67(5): 689-701.
54. Fishman D, Faulds G, Jeffery R, Mohamed-Ali V, Yudkin J S, Humphries S, Woo P. The effect of novel polymorphisms in the interleukin-6 (IL-6) gene on IL-6 transcription and plasma IL-6 levels, and an association with systemic-onset juvenile chronic arthritis. J Clin Invest 1998; 102 (7): 1369-76.
55. Goris A, Epplen C, Fiten P, Andersson M, Murru R, sciacca F L, Ronsse I; jackel S, Epplen J T, Marrosu M G, Olsson T, Grimaldi L M, Opdenakker G, Billiau A, Vandenbroeck K. Analysis of IFN-gamma gene (IFNG) polymorphism in multiple sclerosis in Europe: effect of population structure on association with disease. J Interferon Cytokine Res 1999; 19(9): 1037-1046.
56. Kilpinen S, Hulkkonen J, Wang X Y, Hurme M. The promoter polymorphism of the IL-6 gene regulates IL-6 production in neonates but not in adults. Eur Cytokine Netw 2001; 12 (1): 62-8.
57. Kim J M, Brannan C I, Copeland N G, Jenkins N A, Khan T A, Moore K W. Structure of the mouse IL-10 gene and chromosomal localisation of the mouse and human genes. Journal of Immunology 1992; 148: 3618.
58. Lio D, Scola L, Crivello A, Colonna-Romano G, Candore G, Bonafè M, Cavallone L, Franceschi C, Caruso C. Gender-specific association between −1082 IL-10 promoter polymorphism and longevity. Genes and Immunity 2002; 3: 30-33.
59. Llorente L, Richaud-Patin Y, Couderc J, Alarcon-Segovia D, Ruiz-Soto R, Alcocer-Castillejos N, Alcocer-Varela J, Granados J, Bahena S, Galanaud P, Emilia D. Dysregulation of interleukin-10 production in relatives of patients with systemic lupus erythematosus. Arthritis Rheum 1997; 38(8): 1429-35.
60. Mateo I, Sanchez-Guerra M, Combarros O, Llorca J, Infante J, Gonzalez-Garcia J, del Molino J P, Berciano J. Lack of association between cathepsin D genetic polymorphism and Alzheimer disease in a Spanish sample. Am J Med Genet. 2002; 114 (1): 31-3.
61. Murray R E, McGuigan F, Grant S F, Reid D M, Ralston S H. Polymorphisms of the interleukin-6 gene are associated with bone mineral density. Bone 1997; 21: 89-92.
62. Pallaud C, Stranieri C, Sass C, Siest G, Pignatti F, Visvikis S. Candidate gene polymorphimss in cardiovascular disease: a comparative study of frequencies between a French and an Italian population. Clin Chem Lab Med 2001; 39(2): 146-54.
63. Papassotiropoulos A, Hock C, Nitsch R M. Genetics of interleukin 6: implications for Alzheimer's disease. Neurobiology of Aging 1999; 22: 863-871.
64. Pola R, Flex A, Gaetani E, Dal Lago A, Gerardini L, Pola P, Bernabei R. The −174 G/C polymorphism of the interleukin-6 gene promoter is associated with Alzheimer's disease in an Italian population. Neuroreport 2002; 13: 1645-1647.
65. Poli F, Nocco A, Berra S, Scalamogna M, Taioli E, Longhi, Sirchia G. Allele frequencies of polymorphisms of TNF-α, 11-6, 11-10 and IFNG in an Italian Caucasian population. European journal of Immunogenetics 2002; 29: 237-240.
66. Shibata N, Ohnuma T, Takahashi T, Baba H, Ishizuka T, Ohtsuka M, Ueki A, Nagao M, Arai H. Effect of IL-6 polymorphism on risk of Alzheimer disease: genotype-phenotype association study in Japanese cases. American Journal of Medical Genetics 2002; 114: 436-439.
67. Strauss S, Bauer J, Ganter U, Jonas U, Berger M, Volk B. detection of IL-6 and alpha 2-macroglobulin immunoreactivity in cortex and hippocampus of Alzheimer's disease patients. Lab Invest 1992; 66: 223-30.
68. Terry C F, Loukaci V, Green F R. Cooperative influence of genetic polymorphisms on interleukin 6 transcriptional regulation. J Biol Chem 2000; 275 (24): 18138-44.
69. Vandenabeele P, Fiers W. Is amyloidogenesis during Alzheimer's disease due to an IL-1/IL-6 mediated “acute phase response” in the brain?. Immunol Today 1991; 12:217-19.
70. Vandenbroeck K, Hardt C, Louage j, Fiten P, Jackel S, Ronsse I, Epplen J T, Grimaldi L M, Olsson T, Marrosu M G, Billiau A, Opdenakker G. Lack of association between the interferon regulatory factor-1 (IRF1) locus at 5p31.1 and multiple sclerosis in Germany, northern Italy, Sardinia and Sweden. Genes Immun 2000; 1(4): 290-2.
71. Quintana F J. Carmi P. Cohen I R. DNA vaccination with heat shock protein 60 inhibits cyclophosphamide-accelerated diabetes. Journal of Immunology. 169(10):6030-5, 2002 Nov. 15.
72. Dalpke A H. Opper S. Zimmermann S. Heeg K. Suppressors of cytokine signaling (SOCS)-1 and SOCS-3 are induced by CpG-DNA and modulate cytokine responses in APCs. Journal of Immunology. 166(12):7082-9, 2001 Jun. 15.
73. Yi A K. Yoon J G. Yeo S J. Hong S C. English B K. Krieg A M. Role of mitogen-activated protein kinases in CpG DNA-mediated IL-10 and IL-12 production: central role of extracellular signal-regulated kinase in the negative feedback loop of the CpG DNA-mediated Th1 response. Journal of Immunology. 168(9):4711-20, 2002 May 1.
74. G. Hartmann et al., “CpG DNA: A potent signal for growth, activation, and maturation of human dendritic cells,” Proceedings of the National Academy of Sciences, 96:9305-10, 19975. Hollon T. Coley Toxin's Hidden message: from a 19th century mystery comes a potential new class of drugs: CpG oligonucleotides. The Scientist 15(5): 2001, March 5.
75.
76. Hybridon's press release on May 7, 2003 on data presented during the First Annual Meeting of the Federation of Clinical Immunology Societies held in Boston on May 4 to May 7, 2001, Posters#271 “Modulation of immunostimulatory activity of CpG-oligonucleotides by incorporation of site-specific chemical modifications: Significance of internucleoside negative charge” Poster #229 “Oligonucleotides containing YpG or CpR motifs as potent immunostimulatory agents”, found on: http://www.nrp-euro.com.
77. Du Y. et al. Association of an interleukin 1 alpha polymorphism with Alzheimer's disease. Neurology 55(4): 464-5 2000, Aug. 22.
78. Morris J C, Price A L. Pathologic correlates of nondemented aging, mild cognitive impairment, and early-stage Alzheimer's disease. J Mol Neurosci. 2001; 17(2):101-18.
79. Braak H, Braak E. Frequency of stages of Alzheimer-related lesions in different age categories. Neurobiol Aging. 1997; 18(4):351-7.
80. Savva G M, Wharton S B, Ince P G, Forster G, Matthews F E, Brayne C, et al. Age, neuropathology, and dementia. N Engl J Med 2009; 360(22):2302-9.
81. Bennett D A, Schneider J A, Arvanitakis Z, Kelly J F, Aggarwal N T, Shah R C, et al. Neuropathology of older persons without cognitive impairment from two community-based studies. Neurology 2006; 66(12):1837-44.
82. Schneider J A, Arvanitakis Z, Bang W, Bennett D A. Mixed brain pathologies account for most dementia cases in community-dwelling older persons. Neurology 2007; 69(24):2197-2204.
83. Ferri C P, Prince M, Brayne C, Brodaty H, Fratiglioni L, Ganguli M, et al. Global prevalence of dementia: a Delphi consensus study. Lancet 2005; 366(9503):2112-7.
84. Blackburn E H. Switching and signaling at the telomere. Cell. 2001; 106(6):661-73.
85. Blackburn E H. Structure and function of telomeres. Nature. 1991; 350(6319):569-73.
86. Zakian V A. Telomeres: beginning to understand the end. Science. 1995; 270(5242):1601-7.
87. Blasco M A. Telomeres and human disease: ageing, cancer and beyond. Nat Rev Genet. 2005; 6(8):611-22.
88. Blackburn E H. Telomeres and telomerase: their mechanisms of action and the effects of altering their functions. FEBS Lett. 2005; 579(4):859-62.
89. de Lange T. Protection of mammalian telomeres. Oncogene. 2002 Jan. 21; 21(4):532-40.
90. Allsopp R C, Chang E, Kashefi-Aazam M, Rogaev E I, Piatyszek M A, Shay J W, et al. Telomere shortening is associated with cell division in vitro and in vivo. Exp Cell Res. 1995 September; 220(1):194-200.
91. Kajstura J, Pertoldi B, Leri A, Beltrami C A, Deptala A, Darzynkiewicz Z., et al. Telomere shortening is an in vivo marker of myocyte replication and aging. Am J. Pathol. 2000; 156(3):813-9.
92. Rajaraman S, Choi J, Cheung P, Beaudry V, Moore H, Artandi S E. Telomere uncapping in progenitor cells with critical telomere shortening is coupled to S-phase progression in vivo. Proc Natl Acad Sci USA. 2007; 6; 104(45):17747-52.
93. Vaziri H, Dragowska W, Allsopp R C, Thomas T E, Harley C B, Lansdorp P M. Evidence for a mitotic clock in human hematopoietic stem cells: loss of telomeric DNA with age. Proc Natl Acad Sci USA. 1994; 91(21):9857-60.
94. Hao L Y, Armanios M, Strong M A, Karim B, Feldser D M, Huso D, Greider C W. Short telomeres, even in the presence of telomerase, limit tissue renewal capacity. Cell. 2005; 123(6):1121-31.
95. Blackburn E H. Telomere states and cell fates. Nature. 2000 Nov. 2; 408(6808):53-6.
96. Effros R B. Kleemeier Award Lecture 2008—The Canary in the Coal Mine: Telomeres and Human Healthspan. J Gerontol A Biol Sci Med. Sci. 2009; 64A (5): 511-515.
97. Peisu Zhang, Carolyn Dilley, and Mark P. Mattson. DNA Damage Responses in Neural Cells: Focus on the Telomere Neuroscience. 2007; 145 (4): 1439-1448.
98. Blackburn E H, Greider C W, Szostak J W. Telomeres and telomerase: the path from maize, Tetrahymena and yeast to human cancer and aging. Nat Med. 2006; 12 (10):1133-8.
99. Saretzki G, Von Zglinicki T. Replicative aging, telomeres, and oxidative stress. Ann N Y Acad Sci. 2002; 959:24-9.
100. Kawanishi S, Oikawa S. Mechanism of telomere shortening by oxidative stress. Ann N Y Acad Sci. 2004; 1019:278-84.
101. Wolkowitz O M, Mellon S H, Epel E S, Lin J, Dhabhar F S, Su Y, et al. Leukocyte telomere length in major depression: correlations with chronicity, inflammation and oxidative stress—preliminary findings. PLoS One. 2011; 6 (3):e17837.
102. Aviv A. Telomeres and human aging: facts and fibs. Sci Aging Knowledge Environ. 2004; 2004 (51):pe43.
103. Ragno M, Pianese L, Pinelli M, Silvestri S, Cacchió G, Di Marzio F, et al. Shorter telomeres in patients with cerebral autosomal dominant arteriopathy and leukoencephalopathy (CADASIL). Neurogenetics. 2011; 12 (4):337-43.
104. O'Donovan A, Pantell M S, Puterman E, Dhabhar F S, Blackburn E H, Yaffe K, Cumulative inflammatory load is associated with short leukocyte telomere length in the Health, Aging and Body Composition Study. PLoS One. 2011; 6 (5):e19687.
105. Der G, Batty G D, Benzeval M, Deary I J, Green M J, McGlynn L, McIntyre A, et al. Is telomere length a biomarker for aging: cross-sectional evidence from the west of Scotland? PLoS One. 2012; 7 (9):e45166
106. Epel E S, Merkin S S, Cawthon R, Blackburn E H, Adler N E, Pletcher M J, et al. The rate of leukocyte telomere shortening predicts mortality from cardiovascular disease in elderly men. Aging (Albany N.Y.). 2008; 1 (1):81-8.
107. Cawthon R M, Smith K R, O'Brien E, Sivatchenko A, Kerber R A. Association between telomere length in blood and mortality in people aged 60 years or older. Lancet. 2003; 361(9355):393-5.
108. Willeit P, Willeit J, Mayr A, Weger S, Oberhollenzer F, Brandstatter A, et al. Telomere length and risk of incident cancer and cancer mortality. JAMA. 2010; 304 (1):69-75.
109. Zee R Y, Castonguay A J, Barton N S, Germer S, Martin M. Mean leukocyte telomere length shortening and type 2 diabetes mellitus: a case-control study. Transl Res. 2010; 155(4):166-9.
110. Fitzpatrick A L, Kronmal R A, Gardner J P, Psaty B M, Jenny N S, Tracy R P, et al. Leukocyte telomere length and cardiovascular disease in the cardiovascular health study. Am J Epidemiol. 2007; 165(1):14-21.
111. Rode L, Bojesen S E, Weischer M, Vestbo J, Nordestgaard B G. Short telomere length, lung function and chronic obstructive pulmonary disease in 46,396 individuals. Thorax. 2013; 68(5):429-35.
112. Buckingham E M, Klingelhutz A J. The role of telomeres in the ageing of human skin. Exp Dermatol. 2011; 20(4):297-302.
113. Panossian L A, Porter V R, Valenzuela H F, Zhu X, Reback E, Masterman D, et al. Telomere shortening in T cells correlates with Alzheimer's disease status. Neurobiol Aging. 2003; 24(1):77-84.
114. Honig L S, Schupf N, Lee J H, Tang M X, Mayeux R. Shorter telomeres are associated with mortality in those with APOE epsilon4 and dementia. Ann Neurol. 2006; 60(2):181-7.
115. Grodstein F, van Oijen M, Irizarry M C, Rosas H D, Hyman B T, Growdon J H, et al. Shorter telomeres may mark early risk of dementia: preliminary analysis of 62 participants from the nurses' health study. PLoS One. 2008; 3 (2):e1590.
116. Thomas P, O'Callaghan N J, Fenech M. Telomere length in white blood cells, buccal cells and brain tissue and its variation with ageing and Alzheimer's disease. Mech Ageing Dev. 2008; 129(4):183-90.
117. Jenkins E C, Velinov M T, Ye L, Gu H, Li S, Jenkins E C Jr, et al. Telomere shortening in T lymphocytes of older individuals with Down syndrome and dementia. Neurobiol Aging. 2006; 27(7):941-5.
118. Jenkins E C, Ye L, Gu H, Ni S A, Duncan C J, Velinov M, et al. Increased “absence” of telomeres may indicate Alzheimer's disease/dementia status in older individuals with Down syndrome. Neurosci Lett. 2008; 440(3):340-3.
119. Hochstrasser T, Marksteiner J, Humpel C. Telomere length is age-dependent and reduced in monocytes of Alzheimer patients. Exp Gerontol. 2012; 47(2):160-3.
120. Takata Y, Kikukawa M, Hanyu H, Koyama S, Shimizu S, Umahara T, et al. Association between ApoE phenotypes and telomere erosion in Alzheimer's disease. J Gerontol A Biol Sci Med Sci. 2012; 67(4):330-5.
121. Zekry D, Herrmann F R, Irminger-Finger I, Graf C, Genet C, Vitale A M, et al. Telomere length and ApoE polymorphism in mild cognitive impairment, degenerative and vascular dementia. J Neurol Sci. 2010; 299 (1-2):108-11.
122. Zekry D, Herrmann F R, Irminger-Finger I, Ortolan L, Genet C, Vitale A M, et al. Telomere length is not predictive of dementia or MCI conversion in the oldest old. Neurobiol Aging. 2010; 31(4):719-20.
123. Valdes A M, Deary I J, Gardner J, Kimura M, Lu X, Spector T D, et al. Leukocyte telomere length is associated with cognitive performance in healthy women. Neurobiol Aging. 2010; 31(6):986-92.
124. Martin-Ruiz C, Dickinson H O, Keys B, Rowan E, Kenny R A, Von Zglinicki T. Telomere length predicts poststroke mortality, dementia, and cognitive decline. Ann Neurol. 2006; 60(2):174-80.
125. Yaffe K, Lindquist K, Kluse M, Cawthon R, Harris T, Hsueh W C, et al. Telomere length and cognitive function in community-dwelling elders: findings from the Health ABC Study. Neurobiol Aging. 2011; 32(11):2055-60.
126. Devore E E, Prescott J, De Vivo I, Grodstein F. Relative telomere length and cognitive decline in the Nurses' Health Study. Neurosci Lett. 2011; 492(1):15-8.
127. Insel K C, Merkle C J, Hsiao C P, Vidrine A N, Montgomery D W.

Biomarkers for cognitive aging part I: telomere length, blood pressure and cognition among individuals with hypertension. Biol Res Nurs. 2012; 14(2):124-32.
128. Mather K A, Jorm A F, Anstey K J, Milburn P J, Easteal S, Christensen H. Cognitive performance and leukocyte telomere length in two narrow age-range cohorts: a population study. BMC Geriatr. 2010; 10:62.
129. Shastri A, Bonifati D M, Kishore U. Innate immunity and neuroinflammation. Mediators Inflamm. 2013; 2013:342931.
130. Combs C K, Karlo J C, Kao S C, Landreth G E. beta-Amyloid stimulation of microglia and monocytes results in TNFalpha-dependent expression of inducible nitric oxide synthase and neuronal apoptosis. J. Neurosci. 2001; 21(4):1179-88.
131. Halle A, Hornung V, Petzold G C, Stewart C R, Monks B G, Reinheckel T, et al. The NALP3 inflammasome is involved in the innate immune response to amyloid-beta. Nat. Immunol. 2008; 9(8):857-65.
132. Wyss-Coray T, Yan F, Lin A H, Lambris J D, Alexander J J, Quigg R J, et al. Prominent neurodegeneration and increased plaque formation in complement-inhibited Alzheimer's mice. Proc Natl Acad Sci USA. 2002; 99(16):10837-42.
133. Reale M, Iarlori C, Feliciani C, Gambi D. Peripheral chemokine receptors, their ligands, cytokines and Alzheimer's disease. J Alzheimers Dis. 2008; 14(2):147-59.
134. Flanary B E, Sammons N W, Nguyen C, Walker D, Streit W J. Evidence that aging and amyloid promote microglial cell senescence. Rejuvenation Res. 2007; 10(1):61-74.
135. D'Introno A, Solfrizzi V, Colacicco A M, Capurso C, Amodio M, Todarello O, et al. Current knowledge of chromosome 12 susceptibility genes for late-onset Alzheimer's disease. Neurobiol Aging. 2006; 27(11):1537-53.
136. Guan J Z, Guan W P, Maeda T, Makino N. Effect of vitamin E administration on the elevated oxygen stress and the telomeric and subtelomeric status in Alzheimer's disease. Gerontology. 2012; 58(1):62-9.
137. Rubio-Perez J M, Morillas-Ruiz J M. A review: inflammatory process in Alzheimer's disease, role of cytokines. ScientificWorldJournal. 2012; 2012:756357.
138. Szczepanik A M, Funes S, Petko W, Ringheim G E. IL-4, IL-10 and IL-13 modulate A beta(1-42)-induced cytokine and chemokine production in primary murine microglia and a human monocyte cell line. J. Neuroimmunol. 2001; 113(1):49-62.
139. Strle K, Zhou J H, Shen W H, Broussard S R, Johnson R W, Freund G G, Interleukin-10 in the brain. Crit Rev Immunol. 2001; 21(5):427-49.
140. Arosio B, Trabattoni D, Galimberti L, Bucciarelli P, Fasano F, Calabresi C, Interleukin-10 and interleukin-6 gene polymorphisms as risk factors for Alzheimer's disease. Neurobiol Aging. 2004; 25(8):1009-15.
141. McKhann G, Drachman D, Folstein M, Katzman R, Price D, Stadlan E M. Clinical diagnosis of Alzheimer's disease: report of the NINCDS-ADRDA Work Group under the auspices of Department of Health and Human Services Task Force on Alzheimer's Disease. Neurology. 1984; 34(7):939-44.
142. Masse I, Bordet R, Deplanque D, Al Khedr A, Richard F, Libersa C, et al. Lipid lowering agents are associated with a slower cognitive decline in Alzheimer's disease. J Neurol Neurosurg Psychiatry. 2005; 76(12):1624-9.
143. Miller S A, Dykes D D, Polesky H F. A simple salting out procedure for extracting DNA from human nucleated cells. Nucleic Acids Res. 1988; 16(3):1215.
144. Hultdin M, Gronlund E, Norrback K, Eriksson-Lindstrom E, Just T, Roos G. Telomere analysis by fluorescence in situ hybridization and flow cytometry. Nucleic Acids Res. 1998; 26(16):3651-6.
145. Cawthon R. M. Telomere measurement by quantitative PCR. Nucleic Acids Res. 2002; 30 (10):e47.
146. Ruijter J M, Ramakers C, Hoogaars W M, Karlen Y, Bakker O, van den Hoff M J, et al. Amplification efficiency: linking baseline and bias in the analysis of quantitative PCR data. Nucleic Acids Res. 2009; 37 (6):e45.
147. Corder E H, Saunders A M, Risch N J, Strittmatter W J, Schmechel D E, Gaskell P C Jr, et al. Protective effect of apolipoprotein E type 2 allele for late onset Alzheimer disease. Nat Genet. 1994; 7(2):180-4.
148. Farrer L A, Cupples L A, Haines J L, Hyman B, Kukull W A, Mayeux R, et al. Effects of age, sex, and ethnicity on the association between apolipoprotein E genotype and Alzheimer disease. A meta-analysis. APOE and Alzheimer Disease Meta Analysis Consortium. JAMA. 1997; 278(16):1349-56.
149. Fiala M, Lin J, Ringman J, Kermani-Arab V, Tsao G, Patel A, Lossinsky A S, et al. Ineffective phagocytosis of amyloid-beta by macrophages of Alzheimer's disease patients. J Alzheimers Dis. 2005; 7(3):221-32; discussion 255-62.
150. Trieb K, Ransmayr G, Sgonc R, Lassmann H, Grubeck-Loebenstein B. APP peptides stimulate lymphocyte proliferation in normals, but not in patients with Alzheimer's disease. Neurobiol Aging. 1996; 17(4):541-7.
151. Rolyan H, Scheffold A, Heinrich A, Begus-Nahrmann Y, Langkopf B H, Hölter S M, et al. Telomere shortening reduces Alzheimer's disease amyloid pathology in mice. Brain. 2011; 134 (Pt 7):2044-56.
152. Carcaillon L, Pérès K, Péré J J, Helmer C, Orgogozo J M, Dartigues J F. Fast cognitive decline at the time of dementia diagnosis: a major prognostic factor for survival in the community. Dement Geriatr Cogn Disord. 2007; 23(6):439-45.
153. Soto M E, Andrieu S, Cantet C, Reynish E, Ousset P J, Arbus C, et al. Predictive value of rapid decline in mini mental state examination in clinical practice for prognosis in Alzheimer's disease. Dement Geriatr Cogn Disord. 2008; 26(2):109-16

1-17. (canceled)

18. A method for treating a human subject suspected of being at risk for or having Alzheimer's disease, the method comprising:
analyzing a DNA bearing sample taken from said subject to determine the allelic variants present at one or more of the SNP loci at positions −1082 of the gene encoding IL-10, and detecting the G to A substitution at position −1082,
wherein, if the presence of a polymorphism selected from the group consisting of a G to A substitution at position −1082 of the gene encoding IL-10 is determined to be present in the DNA bearing sample, the human subject is treated by administration of pharmaceutical compositions which directly, as an intermediate, as a pre-cursor or pre-pro-cursor, stimulate the synthesis of IL-10 ab initio or by administration of pharmacological compositions that enhance or inhibit antigen specific production of IL-10.

19. The method according to claim 18 which further comprises analyzing the sample to determine the presence of a −174C allele for the gene encoding IL-6 and Apo-E carrier status, wherein if the presence of a −174C allele for the gene encoding IL-6 is determined to be present in the DNA bearing sample, the human subject is further treated by administration of pharmaceutical compositions which inhibit or block the action of IL-6 in the human subject.

20. A method of treating a human subject suspected of being at risk for or having Alzheimer's disease, the method comprising:
analyzing a DNA sample from said subject to determine the allelic variants present at SNP loci position −1082 of the gene encoding IL-10, and detecting the G to A substitution at position −1082, the presence of a −174C allele for the gene encoding IL-6, and Apo-E 4 carrier status, wherein, if the presence of a polymorphism selected from the group consisting of a G to A substitution at position −1082, the presence of a −174C allele for the gene encoding IL-6, and the presence of a Apo-E 4 allele is determined to be present in the DNA sample, the human subject is treated by administration of pharmaceutical compositions which inhibit or block the action of IL-6 in the human subject and further pharmaceutical compositions which directly, as an intermediate, as a pre-cursor or pre-pro-cursor, stimulate the synthesis of IL-10 ab initio or by administration of pharmacological compositions that enhance or inhibit antigen specific production of IL-10.
